(19) United States
(12) Patent Application Publication
(10) Pub. No.: US 2018/0289385 A1
(43) Pub. Date: Oct. 11, 2018

(54) Surgical Micro-Shears and Methods of Fabrication and Use

(71) Applicant: Microfabrica inc., Van Nuys, CA (US)

(72) Inventors: Gregory P. Schmitz, Los Gatos, CA (US); Ming- Ting Wu, San jose, CA (US); Eric C. Miller, Los Gatos, CA (US); Juan Diego Perea, Campbell, CA (US)

(73) Assignee: Microfabrica inc., Van Nuys, CA (US)

(21) Appl. No.: 15/943,598

(22) Filed: Apr. 2, 2018

Related U.S. Application Data
(63) Continuation of application No. 15/292,029, filed on Oct. 12, 2016, now abandoned.
Application No. 15/292,029 is a continuation-in-part of application No. 13/855,627, filed on Apr. 2, 2013, now abandoned.
(60) Provisional application No. 61/710,608, filed on Oct. 5, 2012.
(60) Provisional application No. 62/385,829, filed on Sep. 9, 2016.

(51) Int. Cl. A61B 17/3201; A61N 1/36; A61B 34/30; A61B 18/14; A61B 17/32; A61B 18/12; A61B 18/00; B33Y 10/00; A61B 17/00; B33Y 80/00; C25D 1/00

(52) U.S. Cl. CPC A61B 17/3201; A61B 2018/00595; A61B 34/30; A61B 18/1447; A61B 17/32002; A61B 18/1206; A61B 2018/1253; A61B 2018/00982; A61B 2018/00839; A61B 2018/00702; A61B 2018/00642; A61B 2018/00607; A61B 2018/00601; B33Y 10/00; A61B 2018/00494; A61B 2017/320032; A61B 2017/00818; A61B 2017/00398; A61B 2017/00269; A61B 2017/00039; A61B 18/1445; A61B 2034/305; B33Y 80/00; A61B 2018/126; A61B 2018/146; A61B 2217/005; A61B 2217/007; C25D 1/003; A61N 1/36

(57) ABSTRACT

Methods and devices are provided for use in medical applications involving tissue removal. One exemplary powered scissors device includes a distal housing having a fixed cutting arm located thereon, an elongate member coupled to the distal housing and configured to introduce the distal housing to a target tissue site of the subject, a rotatable blade rotatably mounted to the distal housing, the rotatable blade having at least one cutting element configured to cooperate with the fixed arm to shear tissue therebetween, a crown gear located at a distal end of an inner drive tube, and a first spur gear configured to inter-engage with the crown gear and coupled with the rotatable blade to allow the crown gear to drive the rotatable blade.

### CROSS REFERENCE TO RELATED APPLICATIONS

This application is a continuation of U.S. application Ser. No. 15/292,029, filed on Oct. 12, 2016, which is a continuation-in-part of U.S. application Ser. No. 13/855,627, filed on Apr. 2, 2013, which claims benefit of U.S. Provisional Application No. 61/710,608, filed on Oct. 5, 2012. The '029 application also claims the benefit of U.S. Provisional Application No. 62/385,829, filed on Sep. 9, 2016.

This application is related to the following U.S. applications: application Ser. No. 15/167,899 filed May 27, 2016; Provisional Application No. 62/167,262 filed May 27, 2015; application Ser. No. 13/843,462 filed Mar. 15, 2013; application Ser. No. 13/535,197 filed Jun. 27, 2012, now U.S. Pat. No. 9,451,977; application Ser. No. 13/388,653 filed Apr. 16, 2012; application Ser. No. 13/289,994 filed Nov. 4, 2011, now U.S. Pat. No. 8,475,483; application Ser. No. 13/007,578 filed Jan. 14, 2011; application Ser. No. 12/491,220 filed Jun. 24, 2009, now U.S. Pat. No. 8,795,278; application Ser. No. 12/490,301 filed Jun. 23, 2009, now U.S. Pat. No. 8,475,458; application Ser. No. 12/490,295 filed Jun. 23, 2009, now U.S. Pat. No. 8,968,346; Provisional Application No. 61/408,558 filed Oct. 29, 2010; Provisional Application No. 61/234,989 filed Aug. 18, 2009; Provisional Application No. 61/075,007 filed Jun. 24, 2008; Provisional Application No. 61/075,006 filed Jun. 23, 2008; Provisional Application No. 61/164,864 filed Mar. 30, 2009; and Provisional Application No. 61/164,883 filed Mar. 30, 2009.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

Embodiments of the present disclosure relate to micro-scale and millimeter-scale tissue debridement devices that may, for example, be used to remove unwanted tissue or other material from selected locations within a body of a patient during a minimally invasive or other medical procedure, and in particular embodiments, multi-layer, multi-material electrochemical fabrication methods that are used to, in whole or in part, form such devices.

### BACKGROUND OF THE INVENTION

Debridement is the medical removal of necrotic, cancerous, damaged, infected or otherwise unwanted tissue. Some medical procedures include, or consist primarily of, the mechanical debridement of tissue from a subject. Rotary debrider devices have been used in such procedures for many years.

Some debrider devices with relatively large dimensions risk removing unintended tissue from the subject, or damaging the unintended tissue. There is a need for tissue removal devices which have small dimensions and improved functionality which allow them to more safely remove only the desired tissue from the patient. There is also a need for tissue removal devices which have small dimensions and improved functionality over existing products and procedures which allow them to more efficiently remove tissue from the patient.

Micro shears or scissors may be used to debride tissue and/or to make cuts into or through tissue. In some procedures using micro shears, tissue on both sides of a cut is preserved and may be sutured or otherwise rejoined together.

The development of micro shears or scissors is an area which can benefit from the ability to produce the devices, or certain parts of the devices, with small or very small dimensions, but with improved performance over existing products and procedures. Some devices with relatively large dimensions risk cutting and/or removing unintended tissue from the subject, or damaging the unintended tissue. There is a need for tissue cutting and/or removal devices which have small dimensions and improved functionality which allow them to more safely cut and/or remove only the desired tissue from the patient. There is also a need for tissue cutting and/or removal devices which have small dimensions and improved functionality over existing products and procedures which allow them to more efficiently cut and/or remove tissue from the patient.

An electrochemical fabrication technique for forming three-dimensional structures from a plurality of adhered layers is being commercially pursued by Microfabrica® Inc. (formerly MEMGen Corporation) of Van Nuys, Calif. under the name EFAB®. This technique, or in some circumstances other material additive techniques, can be used to fabricate parts having very small dimensions as described above.

Various electrochemical fabrication techniques were described in U.S. Pat. No. 6,027,630, issued on Feb. 22, 2000 to Adam Cohen. Some embodiments of this electrochemical fabrication technique allow the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKING™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:

(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, August 1998.

(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.

(3) A. Cohen, “3-D Micromachining by Electrochemical Fabrication”, Micromachine Devices, March 1999.

(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.

(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST '99), June 1999.

(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.

(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.

(8) A. Cohen, “Electrochemical Fabrication (EFAB™)”, Chapter 19 of The MEMS Handbook, edited by Mohamed Gad-El-Hak, CRC Press, 2002.

(9) Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

An electrochemical deposition process for forming multilayer structures may be carried out in a number of different ways as set forth in the above patent and publications. In one form, this process involves the execution of three separate operations during the formation of each layer of the structure that is to be formed:

1. Selectively depositing at least one material by electrodeposition upon one or more desired regions of a substrate. Typically this material is either a structural material or a sacrificial material.

2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.

3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

One method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). In such a process at least one CC mask is used for each unique cross-sectional pattern that is to be plated.

The support for a CC mask may be a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In some implementations, a single structure, part or device may be formed during execution of the above noted steps or in other implementations (i.e. batch processes) multiple identical or different structures, parts, or devices, may be built up simultaneously.

In preparation for performing the selective deposition of the first operation, the conformable portion of the CC mask is placed in registration with and pressed against a selected portion of (1) the substrate, (2) a previously formed layer, or (3) a previously deposited material forming a portion of the given layer that is being created. The pressing together of the CC mask and relevant substrate, layer or material occurs in such a way that all openings, in the conformable portions of the CC mask contain plating solution. The conformable material of the CC mask that contacts the substrate, layer, or material acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied. Further details of material additive processes may be found in the references cited above.

Tissue removal and/or cutting devices are needed which can be produced with sufficient mechanical complexity and a small size so that they can both safely and more efficiently remove tissue from a subject, and remove and/or cut tissue in a less invasive procedure with less damage to adjacent tissue such that risks are lowered and recovery time is improved. Additionally, tissue removal devices are needed which can aid a surgeon in distinguishing between target tissue to be removed and non-target tissue that is to be left intact. It would also be desirable to have tissue ablation and/or cauterization features incorporated directly into such tissue removal devices.

### SUMMARY OF THE INVENTION

The present disclosure relates generally to the field of tissue removal and more particularly to methods and devices for use in medical applications involving tissue removal.

One exemplary embodiment includes a powered scissors device comprising a distal housing, an elongate member, a rotary blade, a crown gear, and a first spur gear. The distal housing has a fixed cutting arm located thereon. The elongate member is coupled to the distal housing and is configured to introduce the distal housing to a target tissue site of the subject. The elongate member comprises an outer tube and an inner drive tube rotatably mounted within the outer tube. The rotatable blade is rotatably mounted to the distal housing and has at least one cutting element configured to cooperate with the fixed arm to shear tissue therebetween. The crown gear is located at a distal end of the inner drive tube. The first spur gear is configured to inter-engage with the crown gear and is coupled with the rotatable blade to allow the crown gear to drive the rotatable blade.

In some embodiments, the rotatable blade has an axis of rotation that is perpendicular to an axis of rotation of the inner drive tube. The rotatable blade may be partially located within a slot formed within the distal housing such that the at least one cutting element is covered by the distal housing during at least half of its rotation about an axis of rotation of the rotatable blade. The rotatable blade may have multiple cutting elements, each of the cutting elements having a cutting edge configured to cooperate with a cutting edge of the fixed arm to shear tissue therebetween. In some embodiments, every cutting edge of the multiple cutting elements of the rotatable blade lies in a common plane.

According to some aspects of the disclosure, the cutting element may be shorter than the fixed arm. In some embodiments, the cutting element has a top side and a bottom side, is flat on the top side, and has a cutting bevel provided along the bottom side. The cutting element may have a cutting edge that is curved, and the fixed arm may have a cutting edge that is curved in the same direction. In some embodiments, the cutting edges of the cutting element and the fixed arm are curved in an outward direction trailing away from a direction of rotation of the cutting element. In some embodiments, the cutting edge of the cutting element has a smaller radius of curvature than a radius of curvature of the cutting edge of the fixed arm. The fixed arm may be provided with one or more radio frequency electrodes.

The present disclosure provides a number of device embodiments which may be fabricated, but are not necessarily fabricated, from a plurality of formed and adhered layers with each successive layer including at least two materials, one of which is a structural material and the other of which is a sacrificial material, and wherein each successive layer defines a successive cross-section of the three-dimensional structure, and wherein the forming of each of the plurality of successive layers includes: (i) depositing a first of the at least two materials; (ii) depositing a second of the at least two materials; and (B) after the forming of the plurality of successive layers, separating at least a portion of the sacrificial material from the structural material to reveal the three-dimensional structure. In some embodiments, the device may include a plurality of components movable relative to one another which contain etching holes which may be aligned during fabrication and during release from at least a portion of the sacrificial material.

Other aspects of the disclosure will be understood by those of skill in the art upon review of the teachings herein. Other aspects of the disclosure may involve combinations of the above noted aspects of the disclosure. These other aspects of the disclosure may provide various combinations of the aspects presented above as well as provide other configurations, structures, functional relationships, and processes that have not been specifically set forth above.

### BRIEF DESCRIPTION OF THE DRAWINGS

FIG. 1 is a top perspective view showing a first exemplary embodiment of a powered scissors device.

FIG. 2 is a bottom perspective view showing the scissors device of FIG. 1.

FIG. 3 is a top plan view showing the scissors device of FIG. 1.

FIG. 4 is a side elevation view showing the scissors device of FIG. 1.

FIG. 5 is a bottom view showing the scissors device of FIG. 1.

FIG. 6 is an exploded view showing the scissors device of FIG. 1.

FIG. 7 is a side elevation view showing the distal housing or lug of the scissors device of FIG. 1.

FIG. 8 is a distal end view showing the distal housing or lug of the scissors device of FIG. 1.

FIG. 9 is a proximal end view showing the distal housing or lug of the scissors device of FIG. 1.

FIGS. 10-22E are various views showing a second exemplary embodiment of a powered scissors device.

FIGS. 23A-23F are side views showing an exemplary tissue cutting procedure.

FIGS. 24-25C are various views of a tissue cutting system having an articulating wrist.

FIGS. 26A-26D are various views of another tissue cutting system having an articulating wrist.

FIGS. 27-32 are various views of third exemplary embodiment of a powered scissors device having a reciprocating blade.

FIG. 33 is an enlarged perspective view showing the distal end of a tissue cutting system employing an endoscope.

FIGS. 34-47 are various views of systems and methods for removing polyps according to aspects of the disclosure.

### DETAILED DESCRIPTION OF PREFERRED EMBODIMENTS

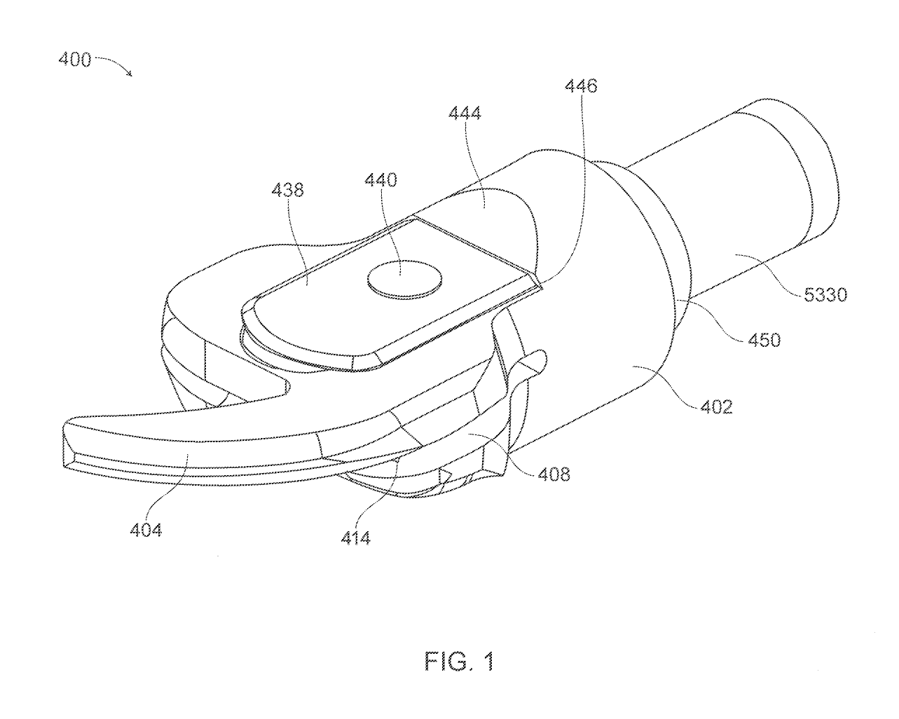

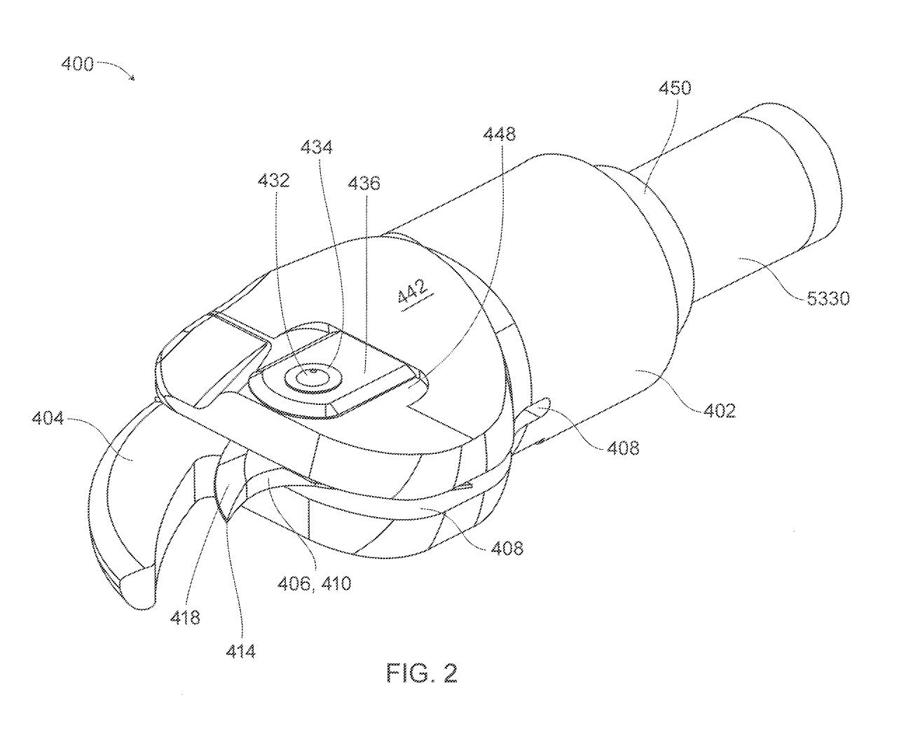

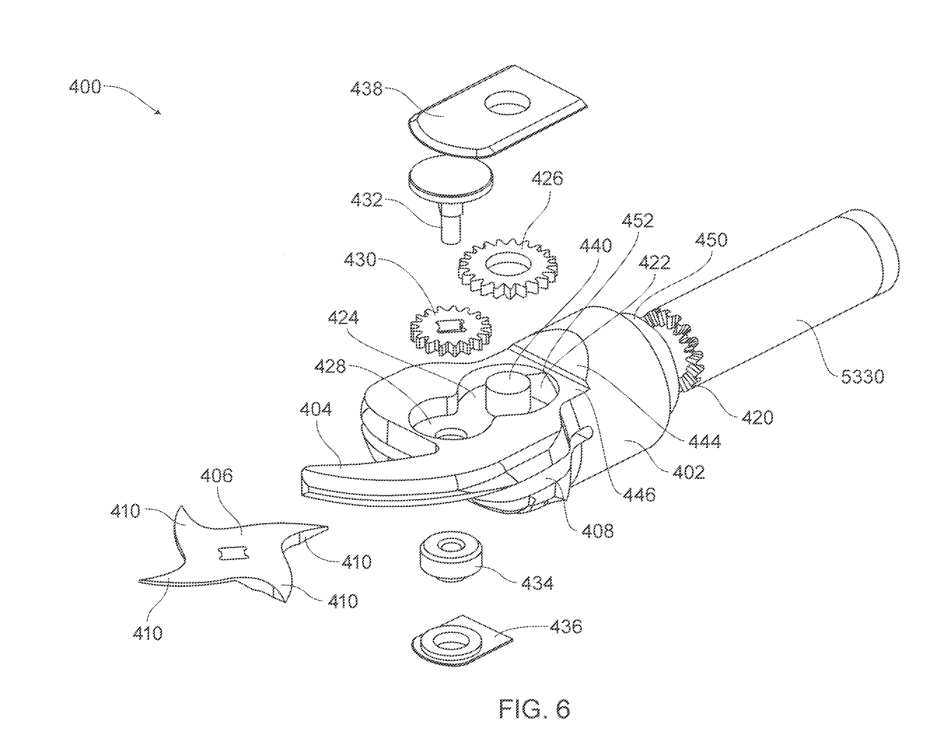

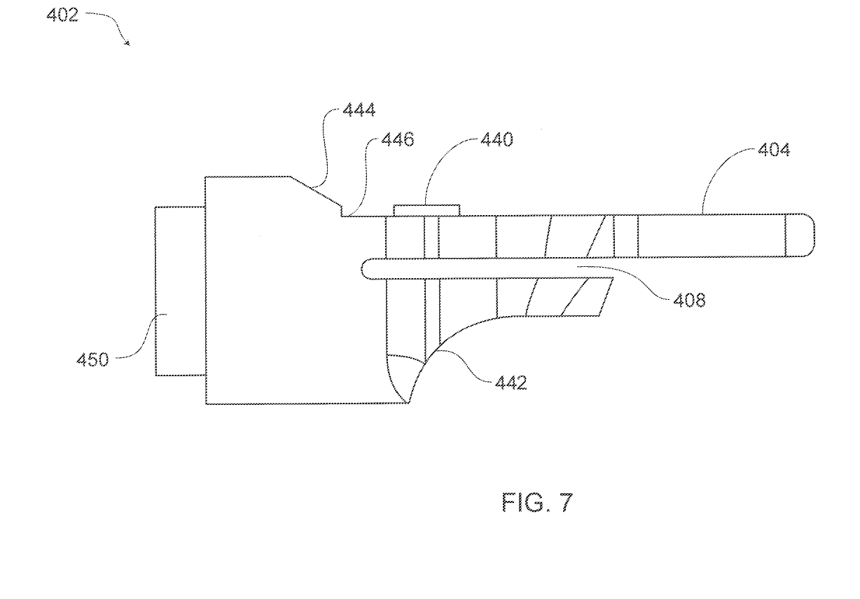

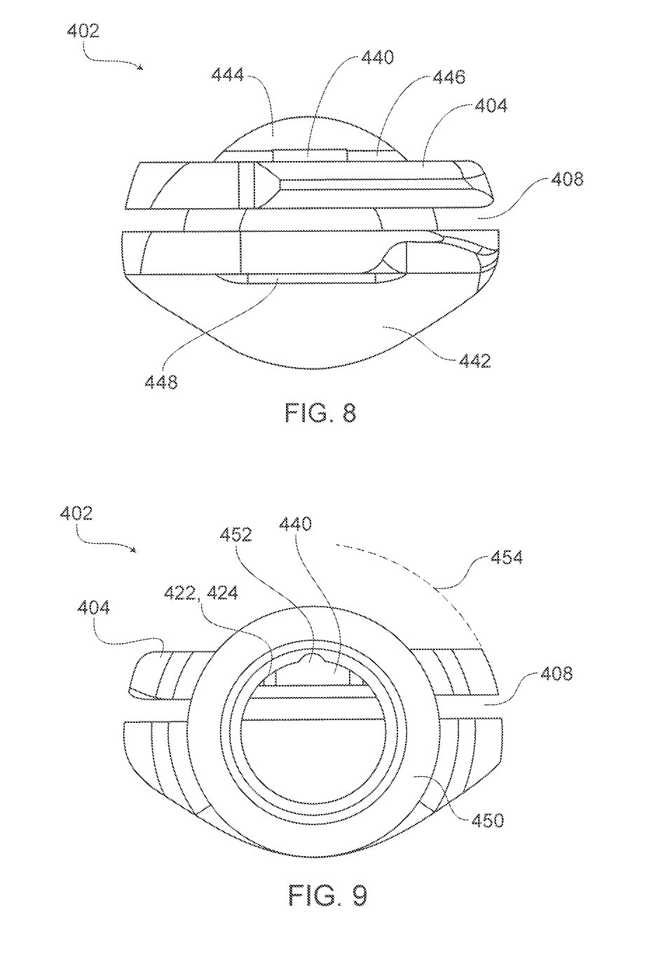

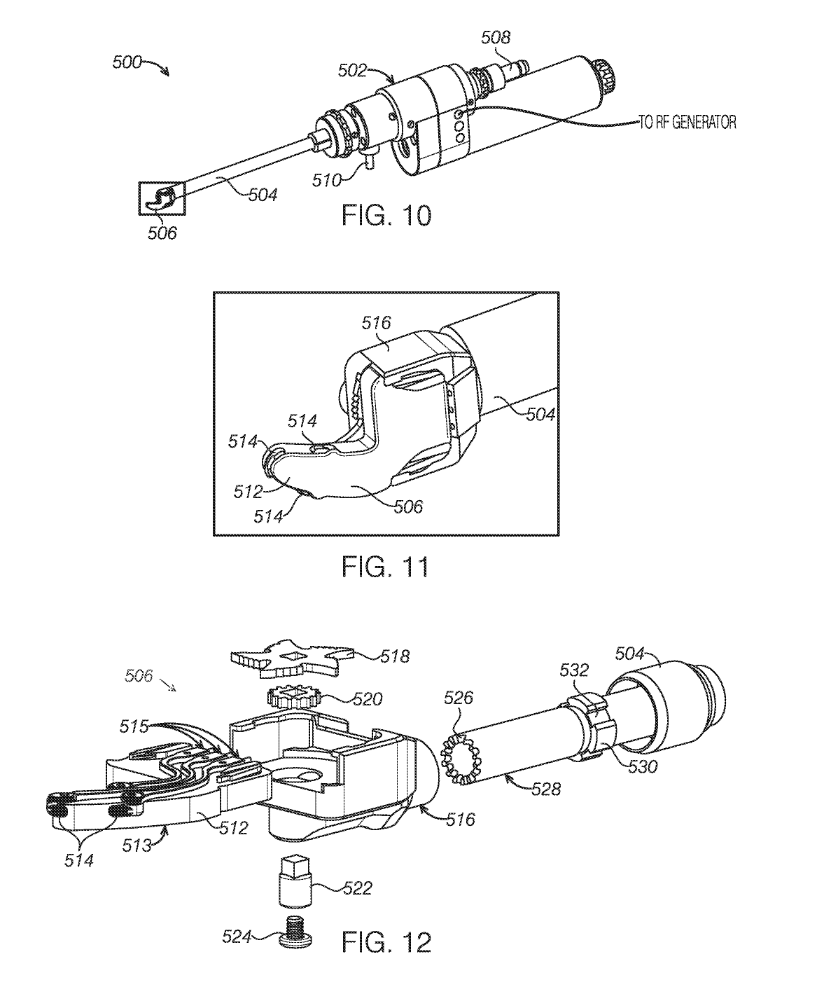

FIGS. 1-9 show a first exemplary embodiment of a tissue cutting device constructed according to aspects of the present disclosure. Device 400 is a powered scissors construct that may be coupled to the distal end of any elongate member configured to introduce the device to a target tissue site of a subject, such as the motorized handpiece 502 shown in FIG. 10, or the fixed or articulating shafts disclosed in U.S. Patent Application Publication 2014/0100558. FIGS. 1 and 2 are top and bottom perspective views, respectively, showing the overall construction of device 400. As shown in these figures, device 400 includes a distal housing or lug 402 provided with a distally extending, arcuate, fixed arm or horn 404. Rotating blade 406 is rotatably mounted within slot 408 that traverses the distal end of lug 402, as best seen in FIG. 7. Blade 406 is provided with four arcuate cutting elements 410 (as best seen in FIG. 6) that capture and shear tissue in turn between each cutting element 410 and fixed arm 404 as blade 406 rotates in the direction shown by Arrow 412. Rotating blade 406 is driven by inner drive tube 5330, as will subsequently be described in detail.

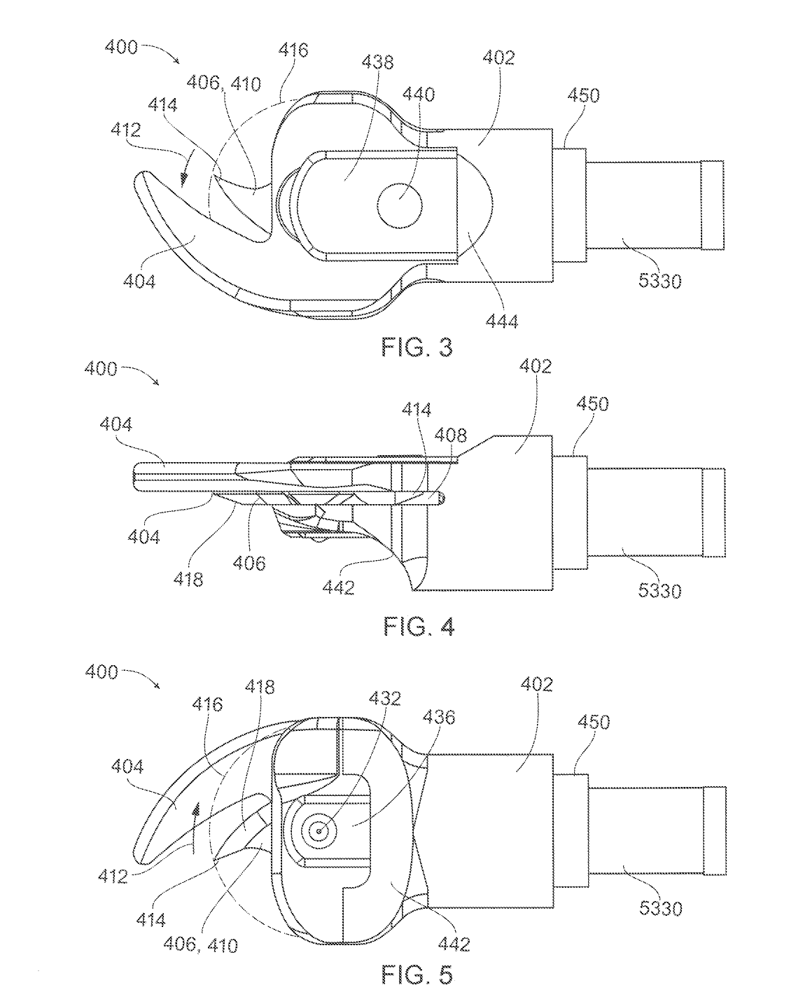

Referring to FIGS. 3-5, top, side and bottom views, respectively, are provided showing device 400 of FIGS. 1 and 2. As can be seen in these drawings, cutting elements 410 of rotating blade 406 are shorter than fixed arm 404. The outer tips 414 of cutting elements 410 travel along circular path 416 depicted by dotted lines in FIGS. 3 and 5. Cutting elements 410 are shielded from adjacent tissue during the majority of their travel around their axis of rotation by the portions of lug 402 above and below slot 408. As best seen in FIGS. 3 and 5, tissue may be cut by device 400 when it enters the space between a cutting element 410 and fixed arm 404, and is then sheared between the two elements as cutting element 410 rotates under fixed arm 404. In this exemplary embodiment, cutting elements 410 are flat on their top side, as shown in FIG. 3, and have a cutting bevel 418 provided along the bottom side of the leading edge, as shown in FIG. 5. The cutting edge of cutting element 410 is curved in the same direction as the cutting edge of fixed arm 404, namely in an outward direction trailing away from the direction of rotation. The cutting edge of cutting element 410 is provided at a slightly tighter radius than that of fixed arm 404 such that the tissue is progressively cut starting at the proximal ends of the cutting edges and moving towards the distal tip 414 of cutting element 410. In this exemplary embodiment, four cutting elements 410 are provided on blade 406, however in other embodiments more or fewer cutting elements may be provided.

Referring to FIG. 6, the drive train components of device 400 are shown. The distal end of inner drive tube 5330 is provided with a crown gear 420. Further details of inner drive tube 5330 and other proximally located drive components are provided in U.S. Patent Application Publication 2014/0100558. When device 400 is assembled, a top portion of crown gear 420 is accessible through opening 422 in lug 402. An annular recess 424 is provided in the top of lug 402 for rotatably receiving a first spur gear 426. Annular recess 424 communicates with opening 422 such that first spur gear 426 can mesh with crown gear 420. Another recess 428 is provided in the top of lug 402 for rotatably receiving a second spur gear 430. When device 400 is assembled, crown gear 420 drives first spur gear 426, which in turn drives second spur gear 430. Spur gears 426 and 430 rotate about parallel axes that are each perpendicular to the central axis of rotation of crown gear 420.

Second spur gear 430 is provided with a square aperture therethrough for receiving drive pin 432. Similarly, blade 406 is provided with a square aperture therethrough. Drive pin 432 passes through second spur gear 430 and blade 406, and its distal end is received within aligner bushing 434. Aligner bushing 434 is received within a circular recess (not shown) in the bottom of lug 402. Drive pin 432 and aligner bushing 434 cooperate to rotatably mount blade 406 in a proper alignment so that it may be driven by second spur gear 430. Lower retainer cap 436 may be provided to captivate aligner bushing 434 within lug 402. Retainer cap 436 may be welded in place on the bottom of lug 402, as shown in FIG. 5. Similarly, upper retainer cap 438 may be welded in place on the top of lug 402 to rotatably captivate drive pin 432 and first and second spur gears 426 and 430 within their respective recesses in lug 402. Upper retainer cap 438 may be provided with a through hole, as best seen in FIG. 6, for engaging with the gear mounting post 440 in the center of annular recess 424.

Referring to FIGS. 7-9, further details of lug 402 are shown. Curved portion 442 may be provided along the bottom of lug 402 to aid in positioning the distal end of device 400 at the target tissue site without damaging tissue. Bevel 444 may be provided along the top of lug 402, and other features may be rounded as shown to prevent device 400 from damaging adjacent tissue. Recess 446 may be provided adjacent to bevel 444 to make a smooth transition between upper retainer cap 438 and bevel 444. Similarly, recess 448 may be provided adjacent to curved portion 442 to make a smooth transition between lower retainer cap 436 and curved portion 442. Boss 450 may be provided at the proximal end of lug 402 for engaging with the distal end of an outer shaft (not shown) of device 400. The outside diameter of lug 402 may be configured to be the same as the outside diameter of the outer shaft to create a smooth transition between the two elements. One or more fluid channels 452 may be provided along the inside diameter of lug 402, as best seen in FIG. 9, to provide cooling, lubrication and or irrigation fluid to the distal end of device 400. As shown, a fluid channel 452 may be aligned with opening 422 in lug 402 for providing fluid directly to spur gears 426 and 430 and to drive pin 432.

In some embodiments, the distal end of device 400 is configured to fit through a 10 mm trocar, endoscope or catheter, as partially depicted by dotted line 454 in FIG. 9. In other embodiments, device 400 is configured to fit through a 5 mm or smaller opening 454.

As shown and described, rotatable blade 406 of exemplary device 400 rotates about an axis that is perpendicular to an axis of rotation of inner drive tube 5330. In other embodiments (not shown), lug 402, crown gear 420 and first spur gear 426 may be configured such that the axis of rotation of rotatable blade 406 is oriented at a different angle with respect to inner drive tube 5330. In some embodiments, the angle between the two axes is 45 degrees. In other embodiments, the two axes are parallel, with the spur gear(s) located outside of the distal tip of the inner drive tube. In some embodiments, the first spur gear may be tilted downward/inward, such that its axis of rotation falls inside the inner drive tube.

Referring to FIGS. 10-23, a second exemplary embodiment of a tissue cutting system constructed according to aspects of the present disclosure is shown and described. As shown in FIG. 10, system 500 includes a motorized handpiece 502, an elongate shaft 504 distally extending from handpiece 502, and a tissue cutting device 506 removably or permanently attached to the distal end of shaft 504. Handpiece 502 may be provided with irrigation port 508 and/or suction/vacuum port 510 for connecting external irrigation and vacuum supplies with the distal tip of system 500 through elongate shaft 504.

Referring to FIGS. 11 and 12, details of tissue cutting device 506 are shown. FIG. 11 is an enlarged perspective view of the distal end of system 500 shown in FIG. 10, and FIG. 12 is an exploded view of the distal end of system 500. Similar in construction and operation to device 400 previously described in reference to FIGS. 1-9, tissue cutting device 506 includes a removable horn assembly 512 with electrodes 514 located thereon. As will be subsequently described in more detail, horn assembly 512 slides into distal housing 516 and locks into place. Removable horn assembly 512 may include a ceramic circuit board 513 with electrical traces 515 formed thereon. Electrodes 514 may be used in monopolar or bipolar configurations, such as for cutting, sealing, coagulating, desiccating, and/or fulgurating tissue, and may be multiplexed to also allow neuro-stimulation and/or tissue sensing, as will be subsequently described in more detail.

As best seen in FIG. 12, device 506 includes a rotary blade 518 and drive gear 520 mounted on drive pin 522. Compression screw 524 threads into drive pin 522 to retain drive pin 522 in place within a central vertical bore through distal housing 516. Drive gear 520 engages with crown gear 526 located at the distal end of driveshaft 528 to allow driveshaft 528 to drive rotary blade 518 through drive pin 522. Bushing 530 may be provided on inner driveshaft 528 to support its rotation with respect to outer shaft 504. Bushing 530 may be provided with one or more through-passages 532 as shown to allow irrigation fluid to flow distally between inner shaft 528 and outer shaft 504. Irrigation fluid may be used to lubricate the drive train of the rotary shears.

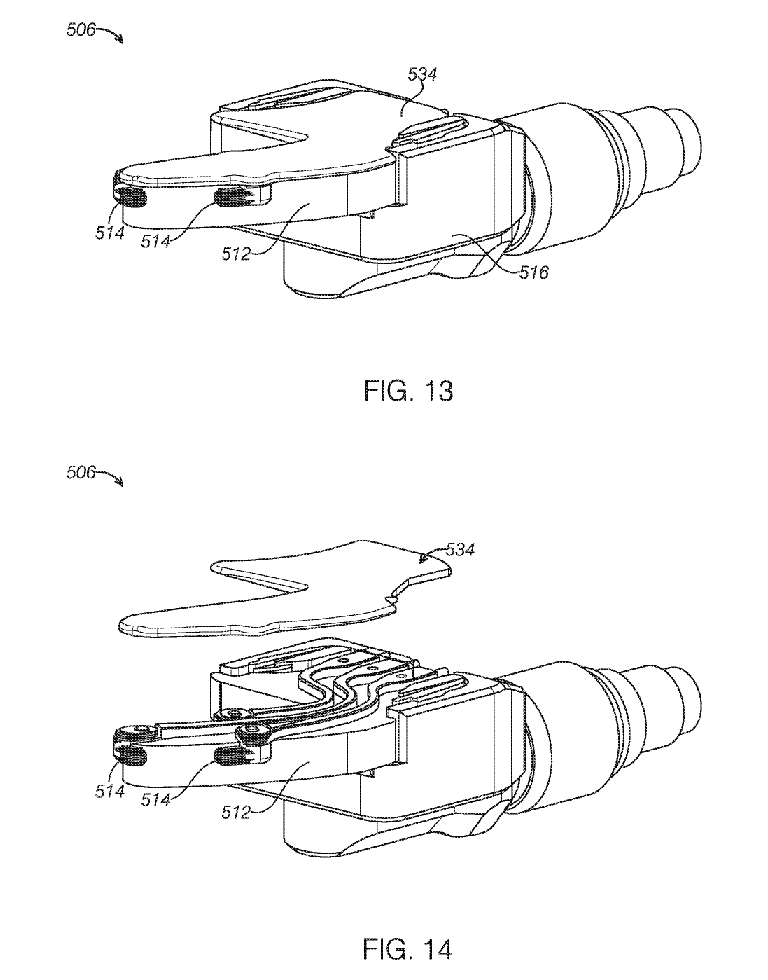

Referring to FIGS. 13 and 14, removable horn assembly 512 may be provided with a dielectric cover 534. Cover 534 protects electrical traces 515 and inhibits electrical shorting/arcing between them. Cover 534 may have a flat top surface as shown and a flat bottom surface (not shown), or a contoured bottom surface that mates with electrodes 514 and electrical traces 515 to further inhibit arcing. Cover 534 may be a separately fabricated piece, such as the plate shown in FIGS. 13 and 14, or it may be a coating formed over the top of substrate 513 and traces 515, such as an insulating epoxy.

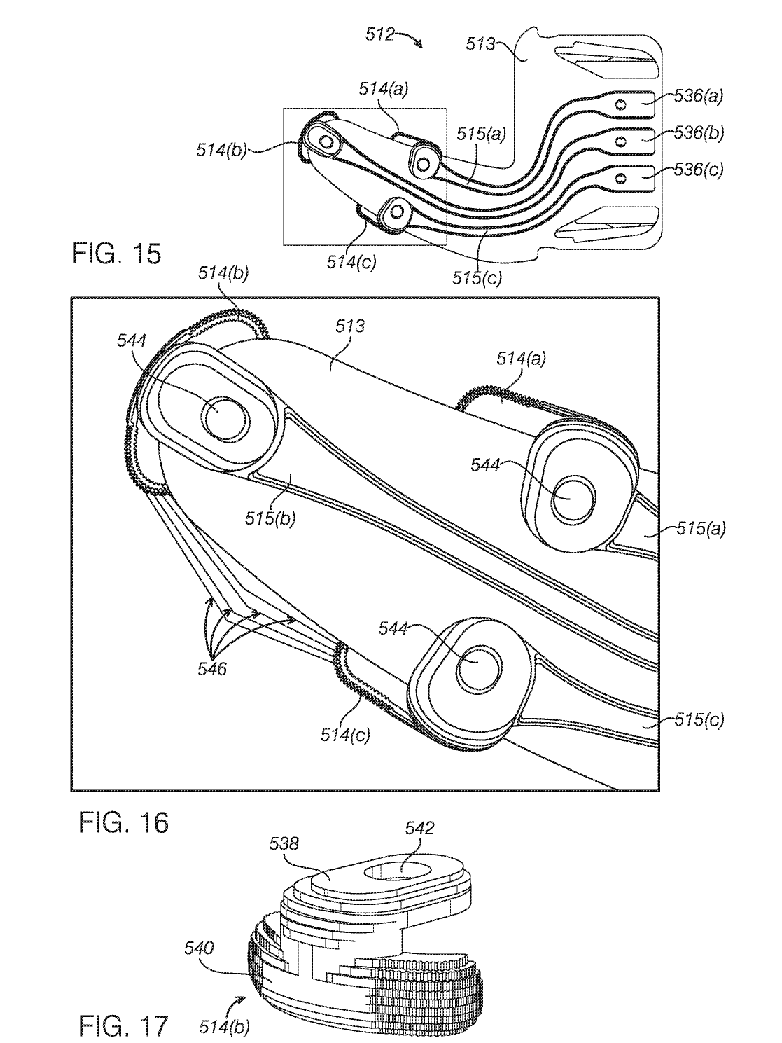

Referring to FIGS. 15-17, details of electrodes 514 and electrical traces 515 are shown. In this exemplary embodiment, removable horn assembly 512 includes three electrical traces 515(a), 515(b) and 515(c) extending along its top surface, three electrodes 514(a), 514(b) and 514(c) located at the distal ends of the electrical traces 515, and three electrical connectors 536(a), 536(b) and 536(c) located at the proximal ends of the electrical traces 515. In some embodiments, electrodes 514, traces 515, and/or connectors 536 are formed in layers on ceramic substrate 513 using an additive process, such as described in co-pending U.S. patent application Ser. No. 15/167,899 filed on May 27, 2016. As will be subsequently described in further detail, connectors 536 may be configured to mate with complementary-shaped connectors or pins located on elongate shaft 504, thereby interconnecting electrodes 514 and electrical traces 515 with a radiofrequency (RF) generator, not shown. Electrodes 514 may be used for tissue sealing, coagulation, neuro-stimulation, tissue sensing, and/or other modes. Irrigation port(s) (not shown) may be provided near or between the electrodes 514 during cauterization or coagulation, as the irrigation fluid can inhibit tissue from sticking to the electrodes 514.

As best seen in FIG. 17, electrodes 514 may each have a top portion 538 and a side portion 540 perpendicular thereto, such that the electrode 514 wraps around a top edge of substrate 513 to extend from its top surface to a side surface. As depicted in FIG. 17, electrodes 514 may be fabricated layer by layer with a material additive process. Because of the three-dimensional nature of electrodes 514, in some embodiments they are fabricated separately from electrical traces 515 and connectors 536 and then assembled on to substrate 513 using a conductive epoxy. An elongated hole 542 may be provided through top portion 538 of electrode 514 for mating with an associated pin 544 formed in trace 515 to ensure the structural and electrical integrity of the connection between electrode 514 and trace 515. As also shown in FIG. 17, layers, serrations, teeth, and/or other texturing features may be formed on the outer working surface(s) of electrodes 514 to increase the overall surface area of electrodes 514 without increasing the size of the electrode's footprint. In some embodiments, the points located on each layer are staggered and/or lined up with points located on adjacent layers. The points may be triangular in shape as shown, square, rectangular, semi-circular, or have other shapes. Adjacent layers may form a stepped, convex curve as shown, or form flat, concave and/or undulating surfaces. With increased surface area, conductivity and effective current density into adjoining tissue increases, thereby reducing arcing and charring of tissue. In some embodiments, it is desirable to increase current density and/or create multiple current paths by texturing the electrodes 514. This can provide a more even distribution of current rather than concentrating the current on a particular edge. Such an arrangement can reduce undesirable sticking and charring of tissue. Is some embodiments, it is desirable to dehydrate the tissue with the electrodes and avoid carbonizing the tissue.

As best seen in FIG. 16, electrodes 514 each extend away from their respective traces 515 in three directions. For example, electrode 514(c) extends from trace 515(c) along the upper surface of substrate 513 (under dielectric cover 534 shown in FIGS. 13 and 14) towards the edge of the upper surface, over the edge and partway down the side of substrate 513, and along the side of substrate 513 towards electrode 514(b) located on the distal tip of substrate 513. Similarly, electrode 514(b) extends from trace 515(b) along the upper surface of substrate 513 (under dielectric cover 534 shown in FIGS. 13 and 14) towards the edge of the upper surface, over the edge and partway down the side of substrate 513, and along the side of substrate 513 towards electrode 514(c). Current paths between electrodes 514(b) and 514(c) are depicted by reference numeral 546 in FIG. 16. In some embodiments, traces 515 may be placed relatively close together without arcing because they are sealed with a dielectric from conductive tissues and bodily fluid. Electrodes 514 are constructed with larger dimensions than those of traces 515 because they are subject to some erosion and/or arcing as the working ends of the electrical circuits. In this embodiment, the terminal ends of traces 515 are kept farther away from each other than the working portions of electrodes 514 to protect the traces from arcing, erosion and/or other potential damage. The working portions of electrodes 514 (e.g. the portions of electrodes 514(b) and 514(c) connected by current paths 546) are extended closer together in three mutually orthogonal directions to further protect traces 515 from damage. In some embodiments (not shown), electrodes 514 extend toward one another and away from their smaller dimensioned respective traces in only two orthogonal directions, or in only one direction.

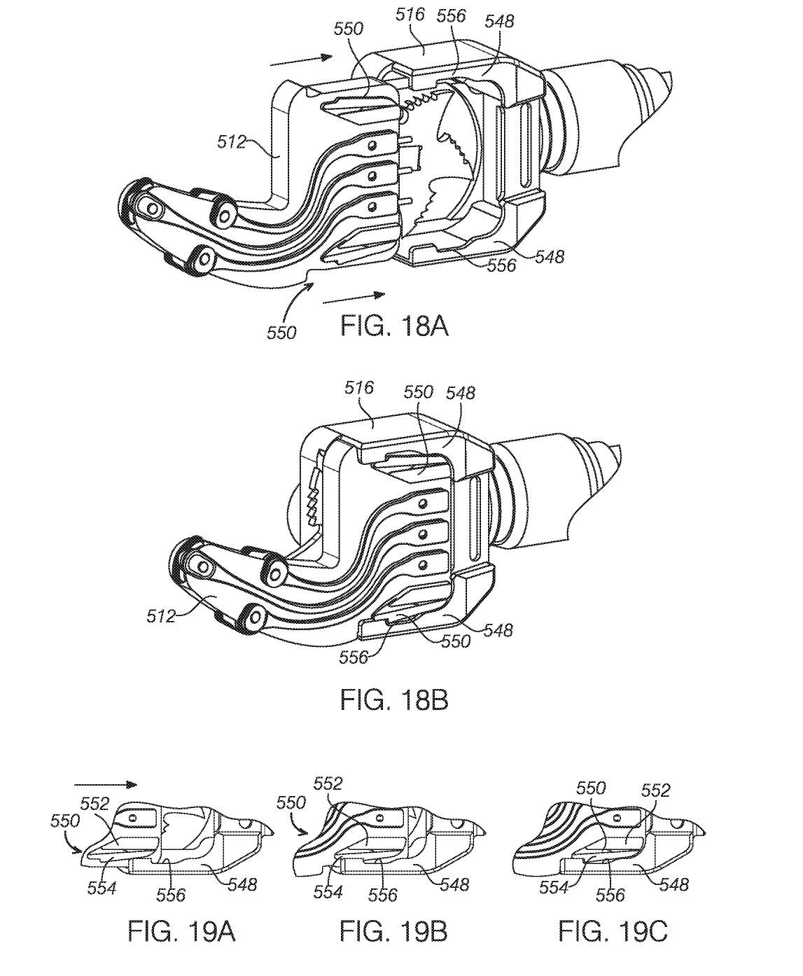

Referring to FIGS. 18A-19C, the removable assembly of horn 512 with housing 516 is shown and described. Housing 516 is provided with a slot for receiving horn assembly 512. The slot is formed in part by overhanging rails 548 on the lateral and proximal portions of housing 516. Locking members 550 may be provided on opposite lateral sides of horn 512 for releasably maintaining horn 512 within the slot of housing 516. Locking members 550 may each be provided with a fixed arm 552 and a movable arm 554 hingedly connected together, such as by a living hinge. With this arrangement, movable arms 554 may flex inwardly as horn 512 is introduced into housing 516, as shown in FIG. 19B. When horn 512 is fully seated in housing 516, movable arms 554 flex outwardly into detents 556 to lock horn 512 into place. To later remove horn 512 from housing 516, movable arms 554 flex inwardly and the horn may be withdrawn. In some implementations, horn assembly 512 is a single use or limited use disposable item. In some implementations, housing 516 is also a single use or limited use disposable item. In some implementations, horn assembly 512 and/or housing 516 may be durable instruments that may be sterilized individually or while remaining assembled together.

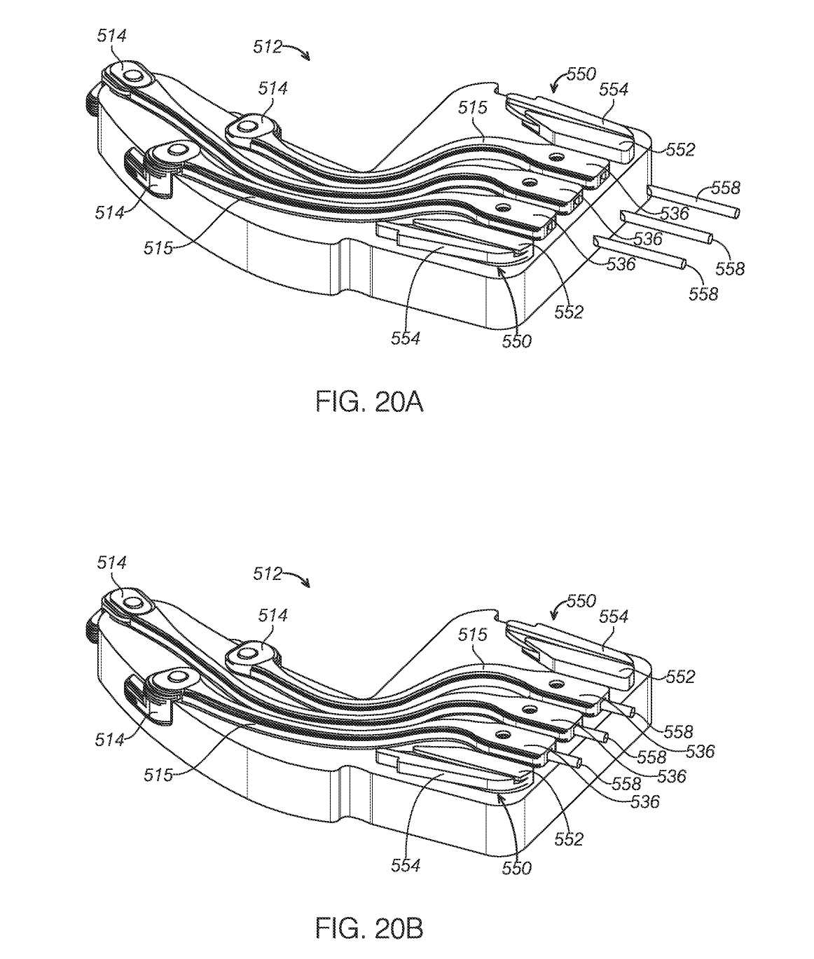

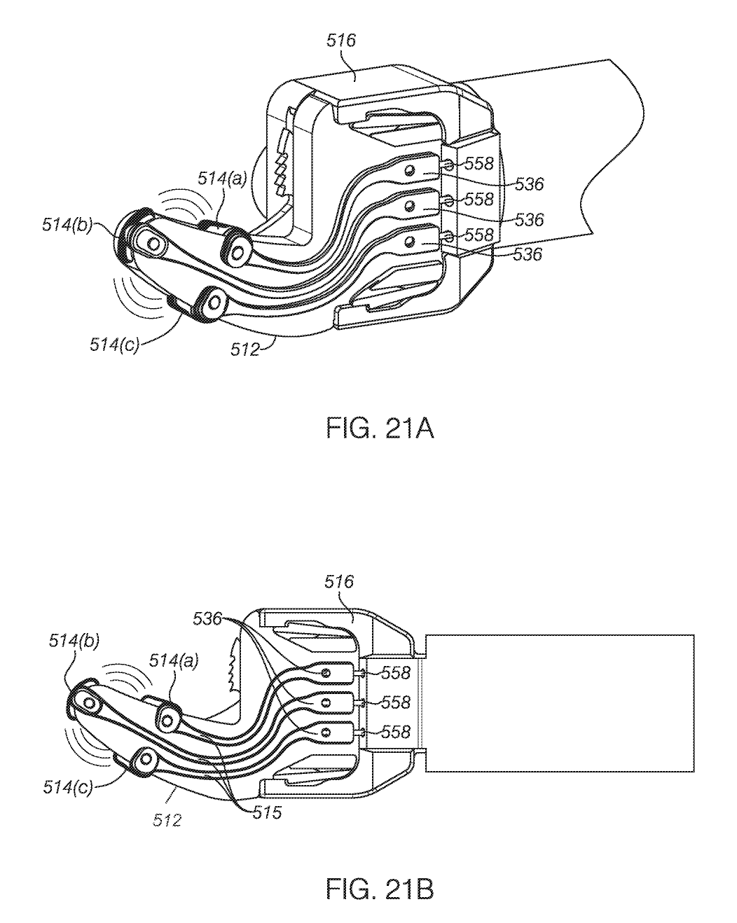

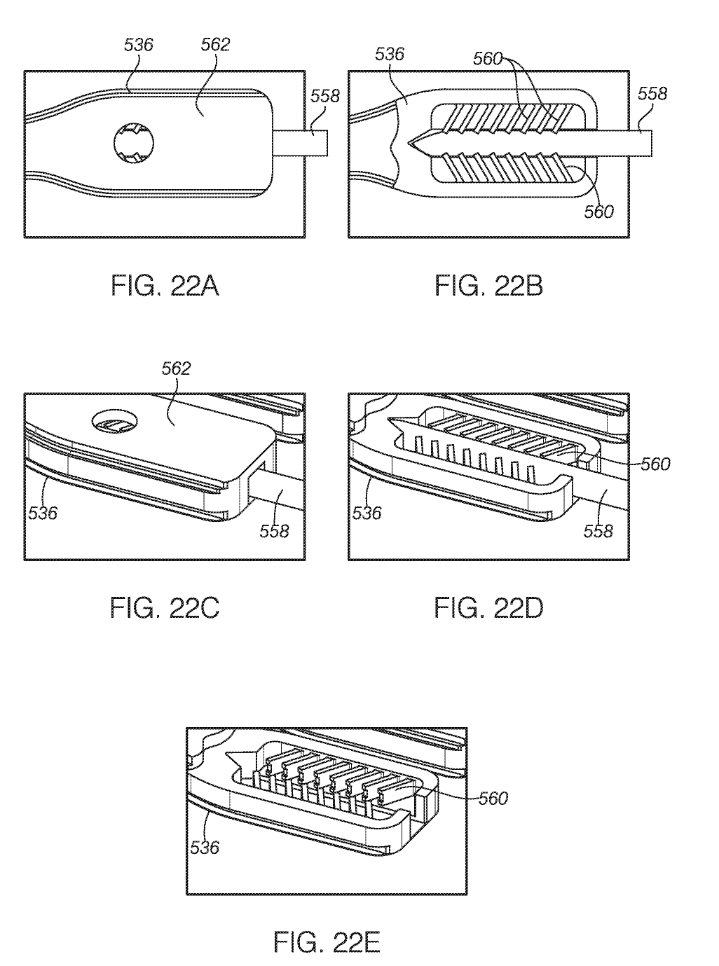

Referring now to FIGS. 20-22, inventive electrical connectors 536 located on horn 512 are shown and described. Connectors 536 may be formed with the same additive process and at the same time with electrical traces 515. Connectors 536 are provided with apertures for receiving mating wires or pins 558. As best seen in FIG. 21A, pins 558 may be located on housing 516, and electrically interconnected through the handheld instrument to external electrical equipment, such as an RF generator and or neural stimulation equipment (not shown). In some embodiments, the center pin 558 electrically connects electrode 514(b) to a RF/neurostimulator multiplexer, while the two outer pins 558 respectively connect electrodes 514(a) and 514(c) to return/common lines of the multiplexer, as shown in FIG. 21B. As shown in FIGS. 22A through 22E, connectors 536 may be internally provided with locking barbs 560. Inwardly extending locking barbs 560 permit pin 558 to be pressed into connector 536 but inhibit the pin's release. The distal ends of locking barbs 560 may be rounded as depicted in FIG. 22E to increase the surface area of engagement between locking barbs 560 and pins 558. A top cover 562 may be provided over the locking barbs 560 to further retain pin 558 within connector 536, as shown in FIGS. 22A and 22C.

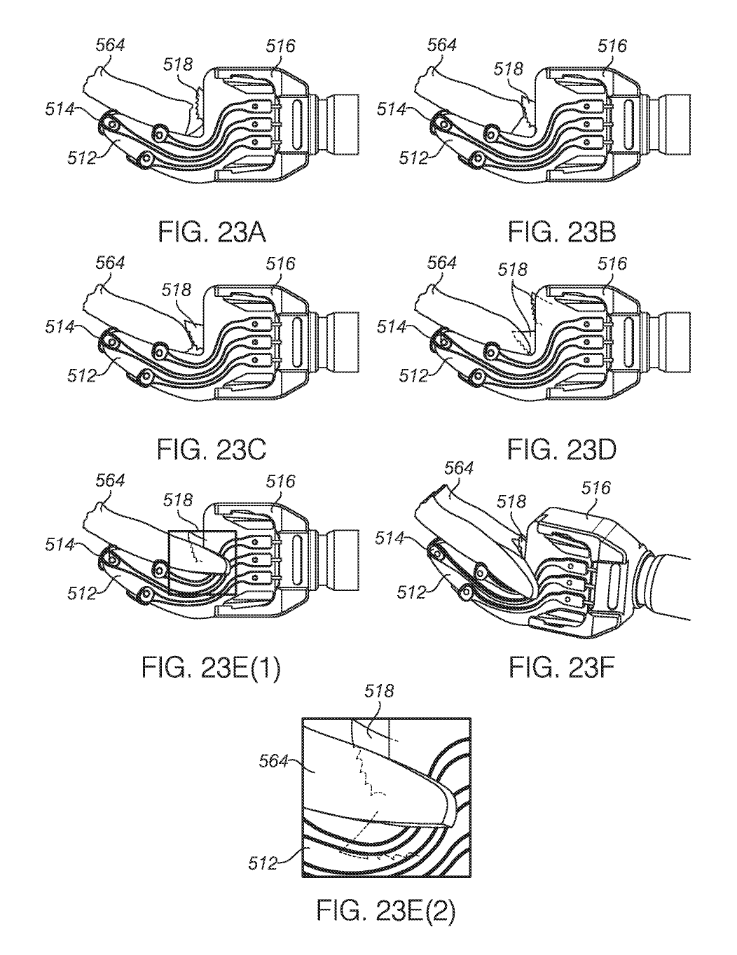

Referring now to the FIGS. 23A-23E, and exemplary tissue cutting process is shown and described. Horn 512 may be used as a probe for creating a safe zone ahead of the tissue cutting. Horn 512 may be slid under a tissue plane so dissection can take place before cutting under tension. The surgeon can then lift up on the cutting device to tension the tissue 564 before actuating the cutting blade 518. As blade 518 rotates, the surgeon can push the instrument forward into the tissue and cut a line through it. In some embodiments, a single, clean line is cut through the tissue without shredding or morselating any of the tissue. FIG. 23A depicts horn 512 after it is slid under tissue 564 and before cutting blade 518 is actuated. FIG. 23B depicts blade 518 starting to rotate and horn 512 being pushed into tissue 564. FIGS. 23C and 23D depict horn 512 being pushed further into tissue 564. As the instrument is pushed still further into tissue 564, the cut tissue splits in half with one half of the tissue sliding along one face of the horn assembly 512 and housing 516 as shown in FIGS. 23E(1), 23E(2) and 23F, and the other half of the tissue sliding along the opposite faces (not shown) of horn assembly 512 and housing 516. During the tissue cutting, electrodes 514 may be used for neuro-stimulation, tissue sensing, and/or coagulation. In some embodiments, actuation of the tissue cutting is performed in a closed loop with the neuro-stimulation. Electromyography (EMG) sensor(s) and system can be incorporated to sense nerve stimulation pulses from electrode(s) 514 and monitor when crucial nerves are in the proximity to the tissue cutting. Power to the cutting motor can be automatically disabled once the cutting is closer to a critical structure than a predetermined threshold.

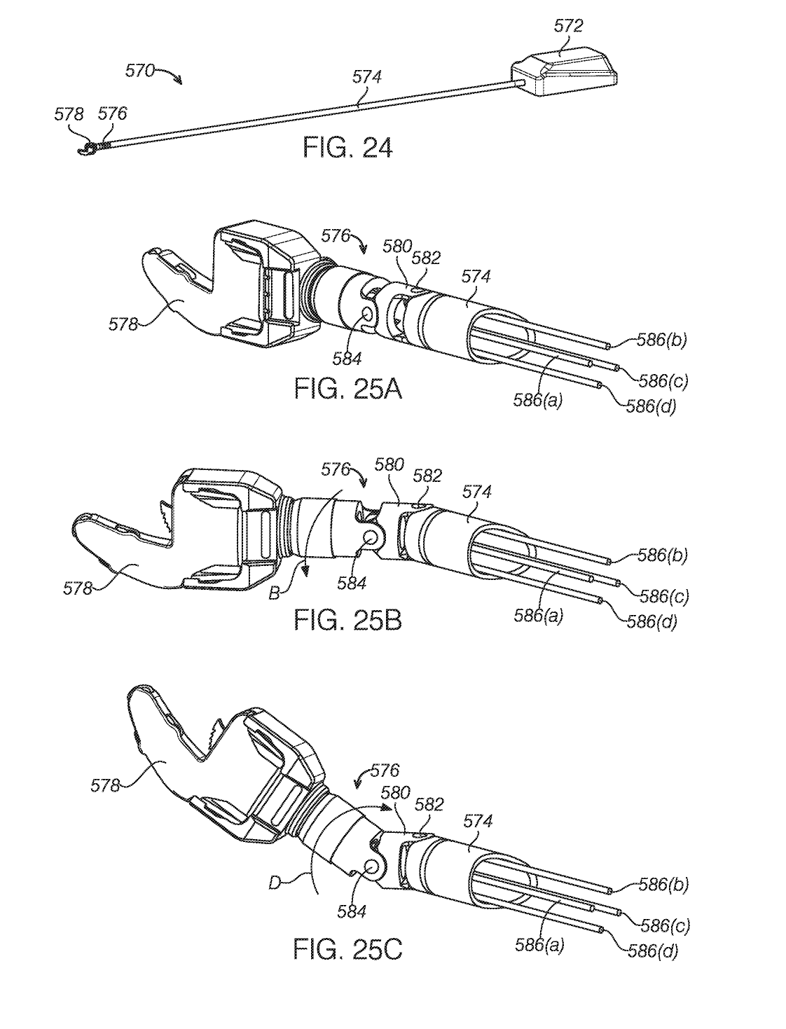

Referring to FIGS. 24 and 25A-25C, an exemplary tissue cutting system 570 is shown and described. As shown in FIG. 24, system 570 includes a control module 572, an elongate shaft 574 extending distally from the control module 572, an articulating wrist 576 located partway along or at the distal end of the elongate shaft 574, and an end effector 578 located at the distal end of articulating wrist 576. Control module 572 may be configured for manual handheld use, or it may be configured to interface with a surgical robot to allow end effector 578 to be operated automatically by a surgical robot and/or by a surgeon using robotic assistance. End effector 578 may be similar or identical to previously described tissue cutting devices 400 or 506 or other tissue cutting devices.

As best seen in FIGS. 25A-25C, articulating wrist 576 includes a central universal joint member 580 that is pivotably connected to elongated shaft 574 with pin (or pins) 582. Central member 580 is also pivotably connected to end effector 578 with pin (or pins) 584. With pin 582 being oriented perpendicular to pin 584, end effector is able to pivot in any direction relative the central axis of shaft 574. Four guide wires 586 may be connected between wrist 576 and controls located in control module 572 (shown in FIG. 24) to allow the wrist to be actuated in any direction by manual or robotic control. For example as shown in FIG. 25B, when guidewire 586(a) which may be connected to central member 580 is pulled proximally in the direction of Arrow A, end effector 578 pivots about pin 582 in the direction of Arrow B. Pulling guidewire 586(c) proximally causes end effector 578 to pivot about pin 582 in the opposite direction. As shown in FIG. 25C, when guidewire 586(b), which may be connected to wrist 576 at a location distal to pin 584, is pulled proximally in the direction of Arrow C, end effector 578 pivots about pin 584 in the direction of Arrow D. Pulling guidewire 586(d) proximally causes end effector 578 to pivot about pin 584 in the opposite direction.

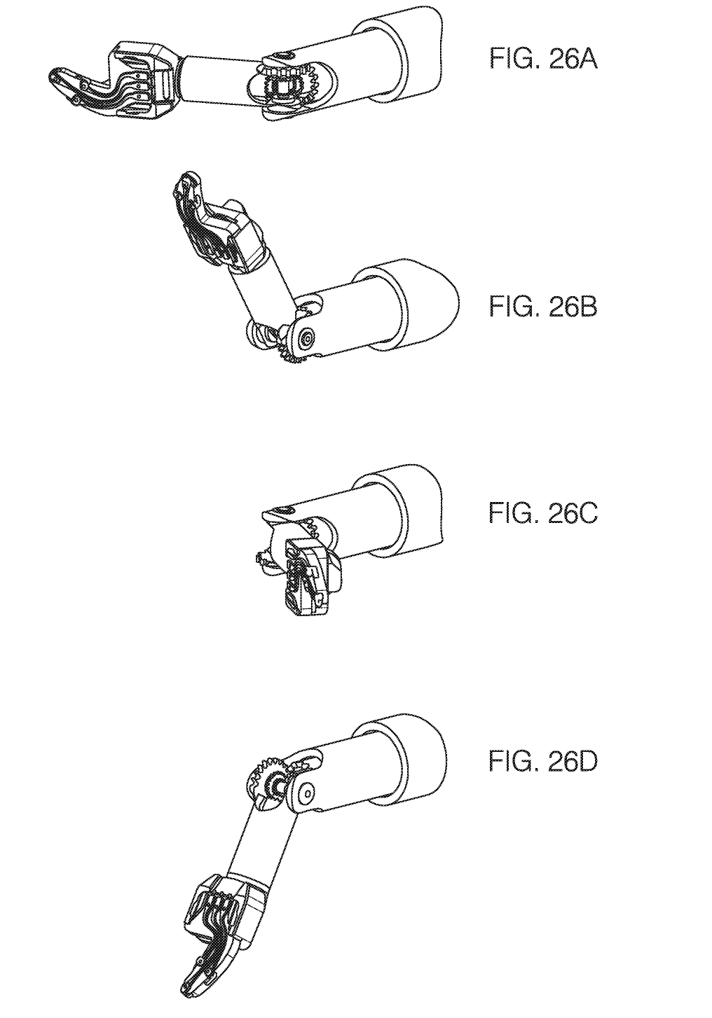

Referring to FIGS. 26A-26D, another construct 588 for articulating end effector 578 is provided. One or more drive tubes 590 nested within elongated shaft 574, each with a crown gear located at its distal end, may be configured to pivot end effector 578 about at least one axis such as 592. For example, end effector 578 may be pivoted right as shown in FIG. 26A, pivoted up as shown in FIG. 26B, pivoted left as shown in FIG. 26C, and pivoted down as shown in FIG. 26D. End effector 578 may also be pivoted and/or rotated about a wrist, elbow and should joint. Further details of this construct are provided in co-pending U.S. Published Application No. 2014/0100558.

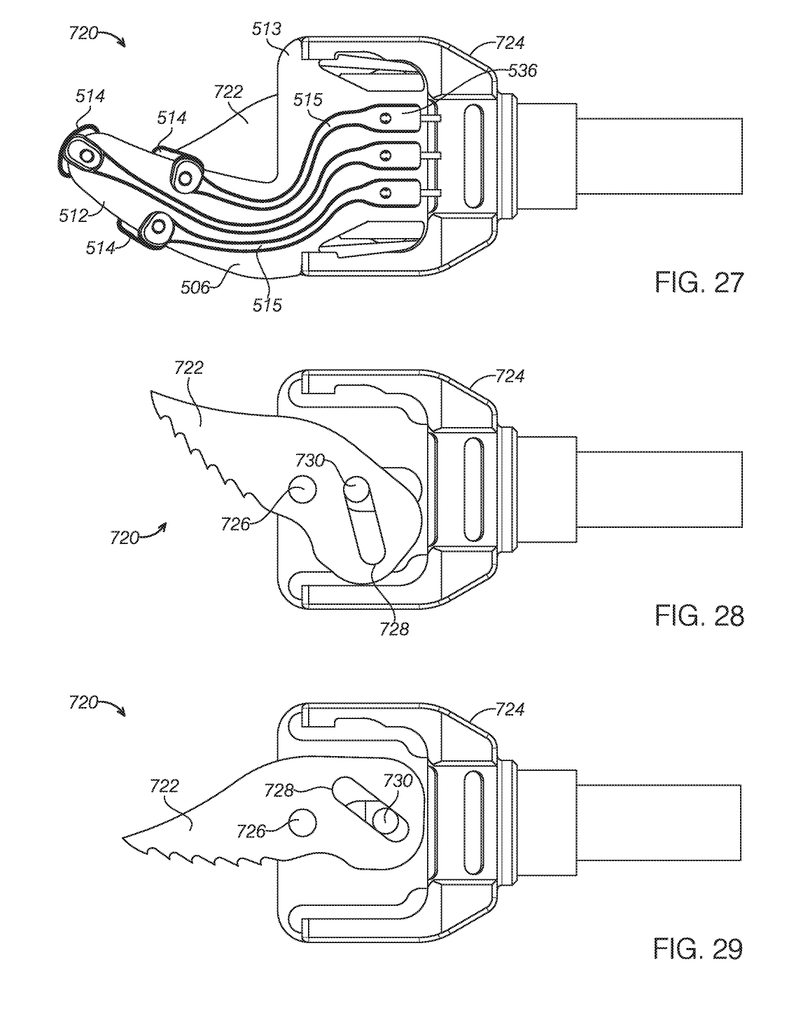

Referring to FIGS. 27-32, a third exemplary embodiment of a tissue cutter device 720 is shown and described. Device 720 is similar to cutting device 506 previously described in reference to FIGS. 10-23 but has a reciprocating blade 722 instead of a rotary cutting blade. As with device 506, device 720 includes a removable horn assembly 506 that slidably mates with housing 724. Horn assembly 506 is provided with the same or similar electrodes 514, electrical traces 515 and electrical connectors 536 on substrate 513, as shown in FIG. 27.

As best seen in FIGS. 28 and 29, reciprocating blade 722 is configured to pivot through a fixed angle range around post 726. FIG. 28 shows blade 722 in an open position and FIG. 29 shows blade 722 in a closed position. As blade 722 pivots from the open position to the closed position it shears tissue against the bottom side of horn 512 (shown in FIG. 27.) In some embodiments, the range of motion of blade 722 between the open and closed positions is about 45 degrees. In some embodiments, blade 722 includes a series of serrations along its leading edge as shown. In other embodiments, blade 722 has a straight leading edge, or a curved leading edge similar to rotary blade 406 shown in FIG. 6.

Reciprocating blade 722 may be provided with a drive slot 728 for slidably receiving drive pin 730. As drive pin 730 is driven distally, blade 722 is pivoted clockwise into the open position, as shown in FIG. 28. When drive pin 730 is driven proximally, blade 722 is pivoted counter-clockwise into the closed position, as shown in FIG. 29.

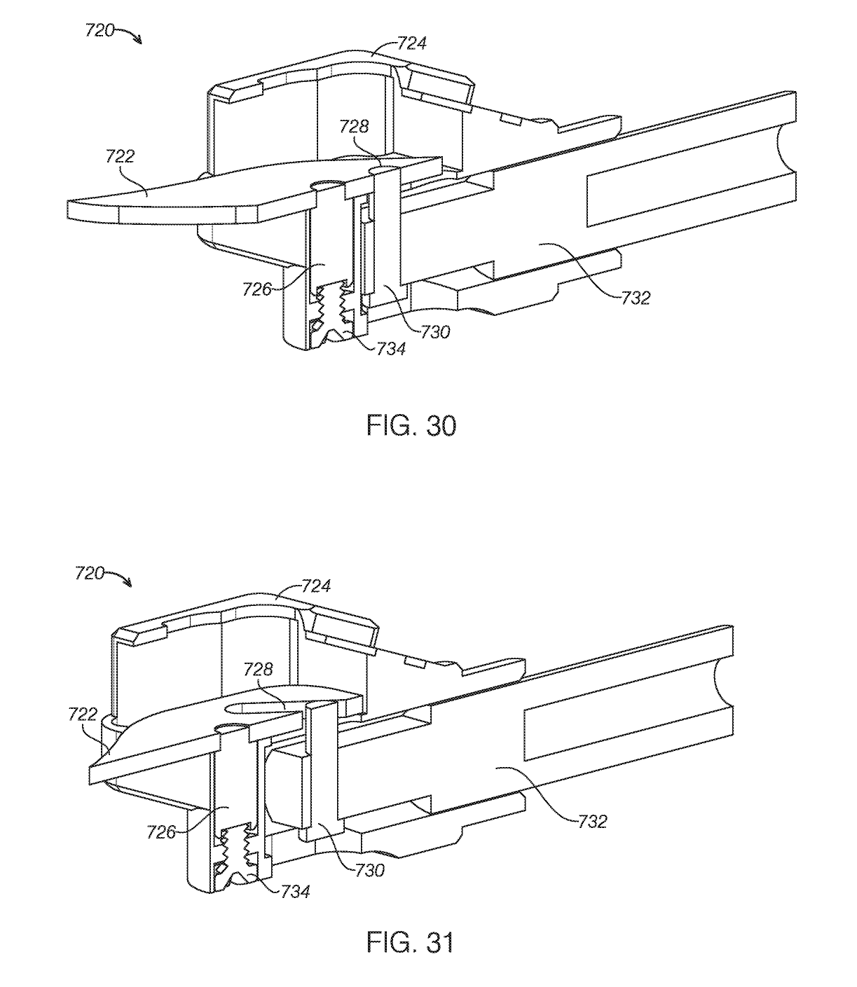

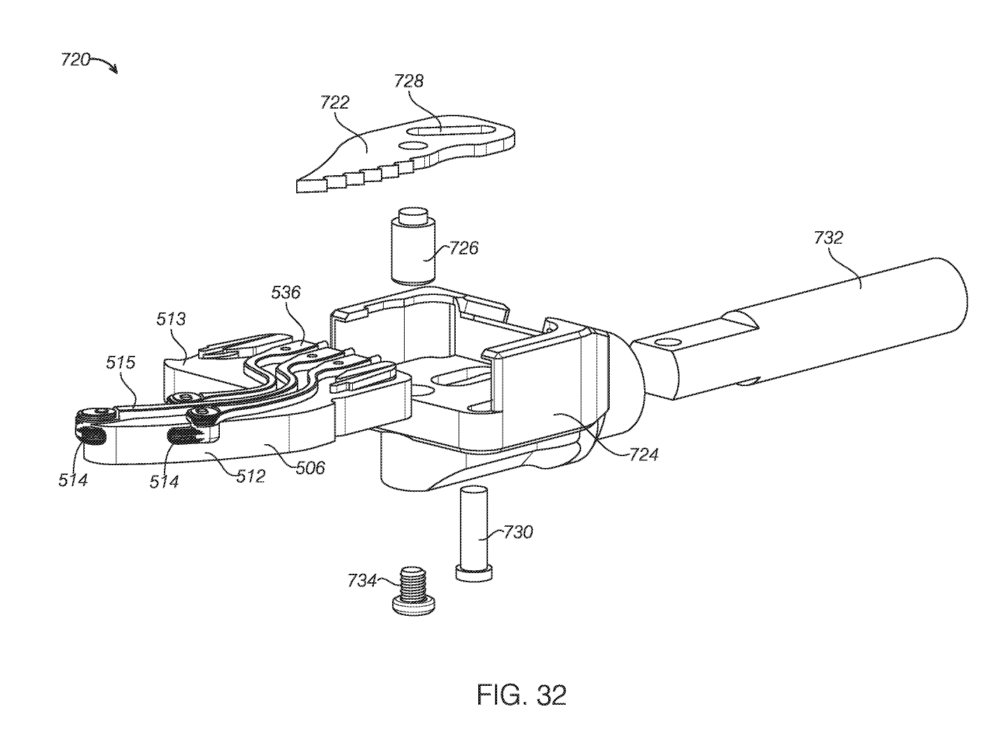

Referring to FIGS. 30 and 31, longitudinal cross-sections of FIGS. 28 and 29 are respectively provided. Drive pin 730 is transversely mounted in reciprocating drive shaft 732. The proximal end of drive shaft 732 is driven by a manually operated trigger, an electric motor, cam, rack and pinion, pneumatics, or other suitable means (not shown) to translate drive shaft 732 distally and proximally to open and close blade 722, respectively. The prime mover that moves shaft 732 may move the shaft in a single direction once with a spring force returning shaft 732 in the opposite direction when released, and/or the prime mover may repeatedly move shaft 732 back and forth, such as when a trigger, button or foot pedal is actuated. As can be seen in FIGS. 30-31, blade pivot post 726 may be secured to housing 724 with a screw 734. Further details of the construction and assembly of tissue cutter device 720 are shown in the exploded diagram of FIG. 32.

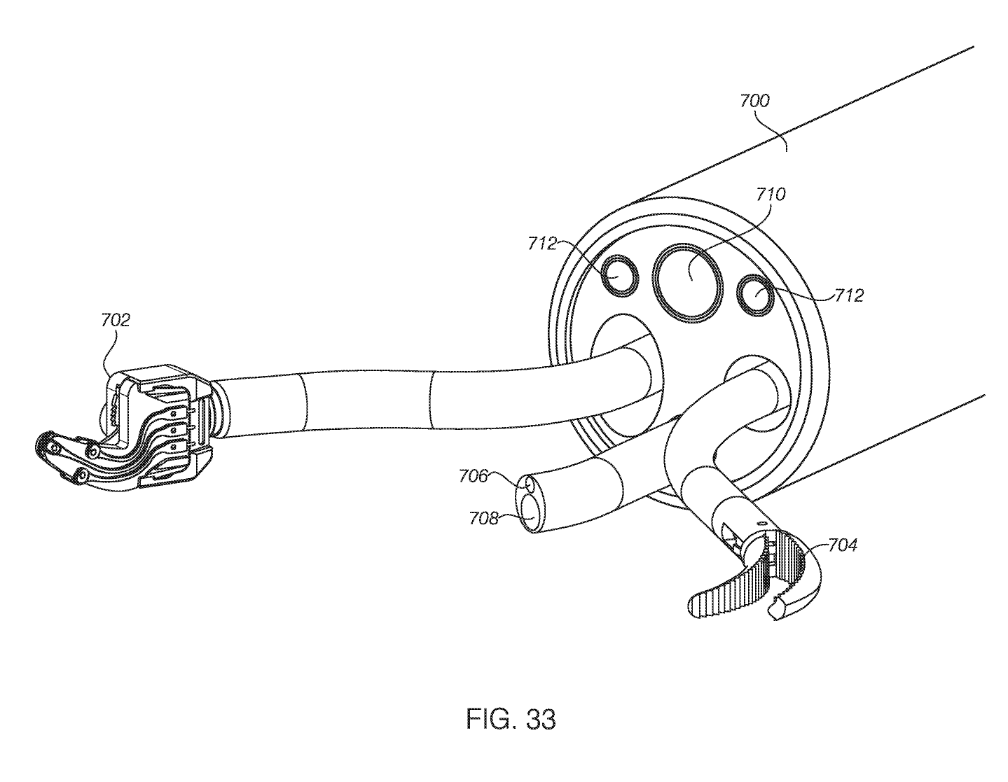

Referring to FIG. 33, an exemplary embodiment is provided with a multi-channel endoscope 700 to introduce a micro powered shear 702 into a target tissue site. Powered shear 702 may be similar or identical to powered shears disclosed herein and may be provided with sections that articulate or bend. For example, powered shear 702 may be provided with articulated joints such as those shown in FIGS. 24-26 so that the distal end of powered shear 702 may be translated and pivoted in three dimensions. Additional movement may come from moving the elongated shaft of powered shear 702 in and out of the endoscope bore and rotating the elongated shaft relative to the endoscope. Powered shear 702 may also be provided with electrodes multiplexed for coagulation and neuro-stimulation. In some embodiments, endoscope 700 is 50 French in size and includes ports for introducing a flexible or articulating shaft grasper 704, irrigation 706, suction 708, a camera 710 and illumination 712. With both the shear 702 and grasper 704 being capable of articulating laterally away from the longitudinal centerline of the endoscope 700 and camera 710 as shown, target tissue may be manipulated by both shear 702 and grasper 704 at the same time from generally opposite lateral sides. The distal tips of shear 702 and grasper 704 may extend back towards each other rather than remaining completely parallel. The powered shears disclosed herein may be used with colonoscopes, arthroscopes, laparoscopes, or other types of endoscopes.

Various embodiments of tissue cutters as described herein may be used with or without an endoscope in the debulking of neuro tumors, prostatectomies, internal mammary artery takedown procedures, facial reconstructive surgeries, carpal tunnel surgeries, submucosa resection of colon polyps (such as the removal at the root base for full biopsy), and other surgical procedures. Further details of an exemplary submucosa colon polyp or tumor resection are provided below.

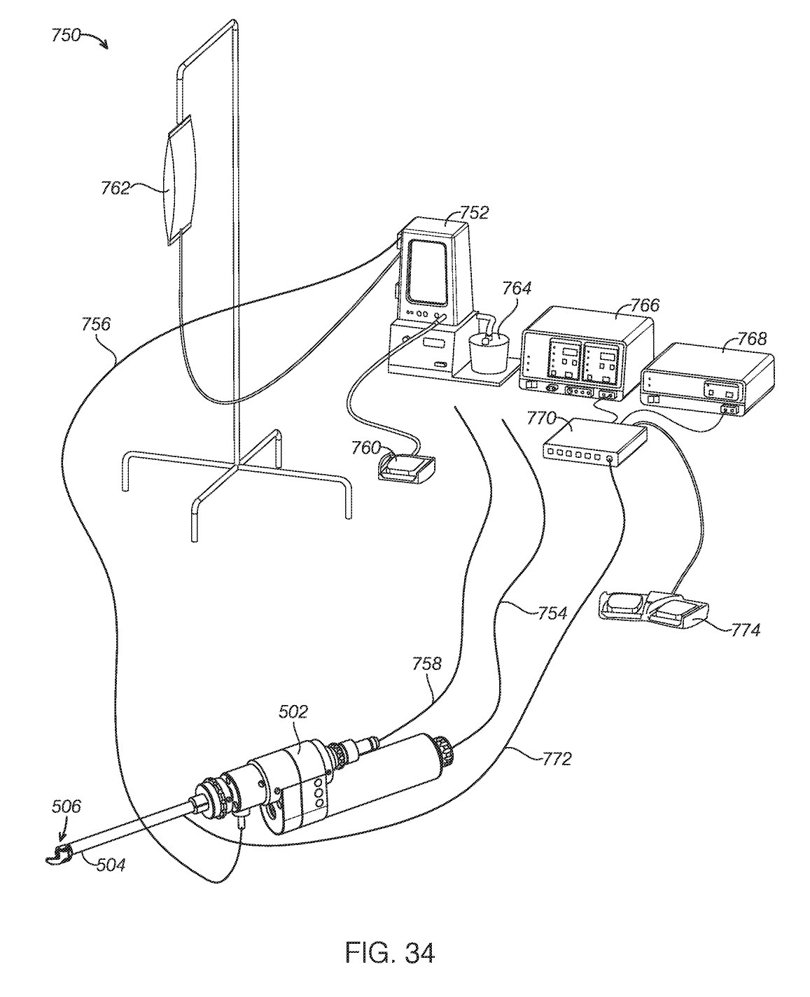

Referring to FIGS. 34-47, exemplary systems and methods for submucosa colon polyp or tumor resection are shown and described. As depicted in FIG. 34, such a system 750 may include a tissue cutting device 506 attached to motorized handpiece 502 through an elongate shaft 504, as previously described in reference to FIG. 10. Elongate shaft 504 may include straight and/or curved sections and may include rigid, flexible, articulating and/or steerable sections. Handpiece 502 in turn may be connected to a user interface control box 752 with motor control cable 754, irrigation line 756, and vacuum line 758. In some embodiments (not shown), the handpiece may be connected to control box 752 with a flexible drive shaft instead of electric motor control cable 754 so that the tissue cutter drive motor may be located in control box 752 instead of in handpiece 502. This relocation of the motor may be useful in reducing the weight, size, complexity and/or cost of the handpiece, and in some embodiments make the handpiece a disposable item. In other embodiments, a pneumatic motor may be located in the hand piece instead of an electric motor, and a pneumatic line instead of an electric cable may be used to connect the handpiece to the control box.

User interface control box 752 may be provided with a foot petal 760 to turn the tissue cutting device drive motor on and off, adjust its speed, and/or reverse its direction of rotation. A pole mounted saline bag 762 may be provided as an irrigation fluid source and connected to control box 752 to control the irrigation provided at tissue cutting device 506. An aspirated material collection bin 764 may also be connected to control box 752 so that the tissue removed through vacuum line 758 can be observed, its volume and/or weight can be measured, and it can be biopsied.

System 750 may include a radio-frequency (RF) electro-surgical box 766 and a neuro-stimulation box 768 as shown in FIG. 34. As previously described, RF box 766 may be interconnected with the electrodes on tissue cutting device 506 to cauterize, coagulate or for otherwise tissue sealing or necrosis at the target site of the patient. As also previously described, neuro stim box 768 may be interconnected with the electrodes on tissue cutting device 506 as a safety measure to help ensure non-target tissue is not cut during the surgical procedure. RF box 766 and neuro stim box 768 may be connected to multiplexer 770 so that only one of the boxes is connected to the cutting device electrodes at any one time. Multiplexer 770 may be connected with handpiece 502 through cable 772, and may be controlled with foot petals 774.

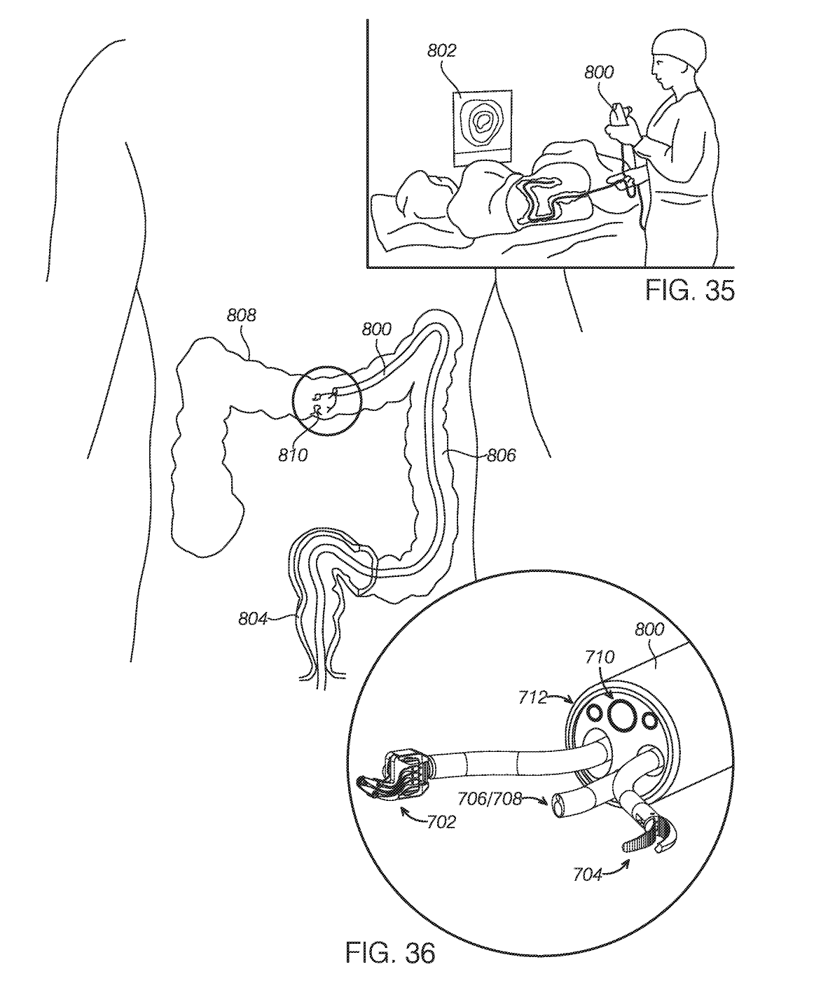

Referring to FIGS. 35-36, the use of previously described system 750 in conjunction with a colonoscope 800 is shown and described. It should be noted that the tissue cutting instrument shown in FIG. 34 may be used with an endoscope or independent from an endoscope. As previously described in reference to FIG. 33, the elongate shaft 504 of the instrument (shown in FIG. 34) may be passed through one lumen of a colonoscope or other endoscope such that the tissue cutting device 506 protrudes from the distal end of the scope and the handpiece 502 resides near the proximal end of the scope. The various components extending from the distal end of the scope may be steerable to allow the surgeon to accomplish tasks requiring a high level of dexterity.

As shown in FIG. 35, the colonoscope 800 may be inserted into a patient's lower gastrointestinal tract through the anus. FIG. 35 depicts the distal end of colonoscope 800 being located at the bottom of the ascending colon. In some procedures, powered shears 702 may be placed within the colonoscope 800 before they are inserted into the patient's body together. In other procedures, colonoscope 800 may be placed first and then shears 702 inserted through the colonoscope. As shown in FIG. 35, the surgeon may be viewing imagery taken by camera 710 at the distal end of the colonoscope (see FIG. 33) on a display 802 as colonscope 800 is advanced through the colon.

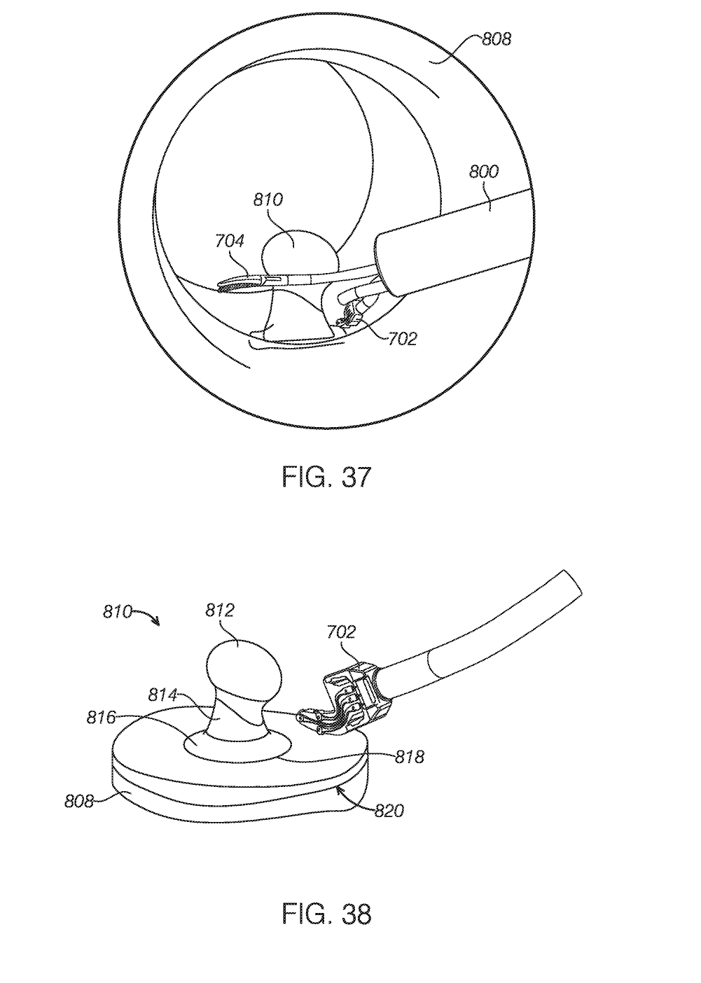

Referring to FIG. 36, colonoscope 800 is depicted traveling through the rectum 804, descending colon 806 and partway across the transverse colon 808. A polyp, tumor, or other tissue of interest 810 is depicted on the lower interior wall of the transverse colon. FIG. 37 is an enlarged view of a portion of FIG. 36 showing polyp 810 being approached from opposite sides by micro-shears 702 and graspers 704 protruding from the distal end of colonoscope 800. FIG. 38 depicts the anatomy of polyp 810 and for clarity shows micro-shears 702 without grasper 704. Exemplary polyp 810 includes a bulbous head portion 812, a reduced diameter body portion 814, an outwardly sloping base portion 816, and a root portion 818 that extends into the submucosa layer 820 of the wall of intestine 808.

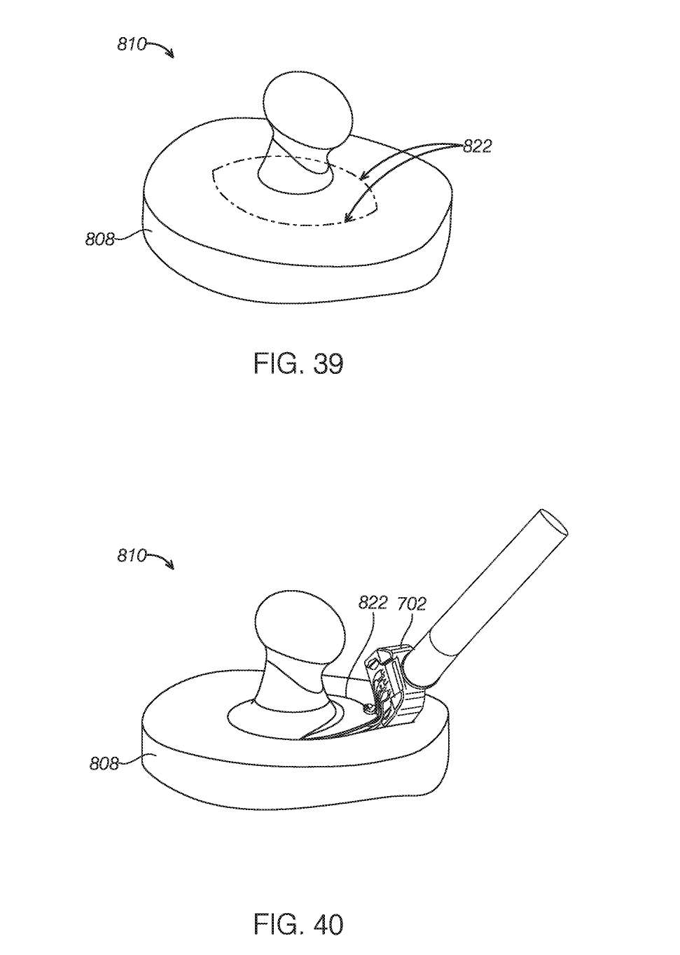

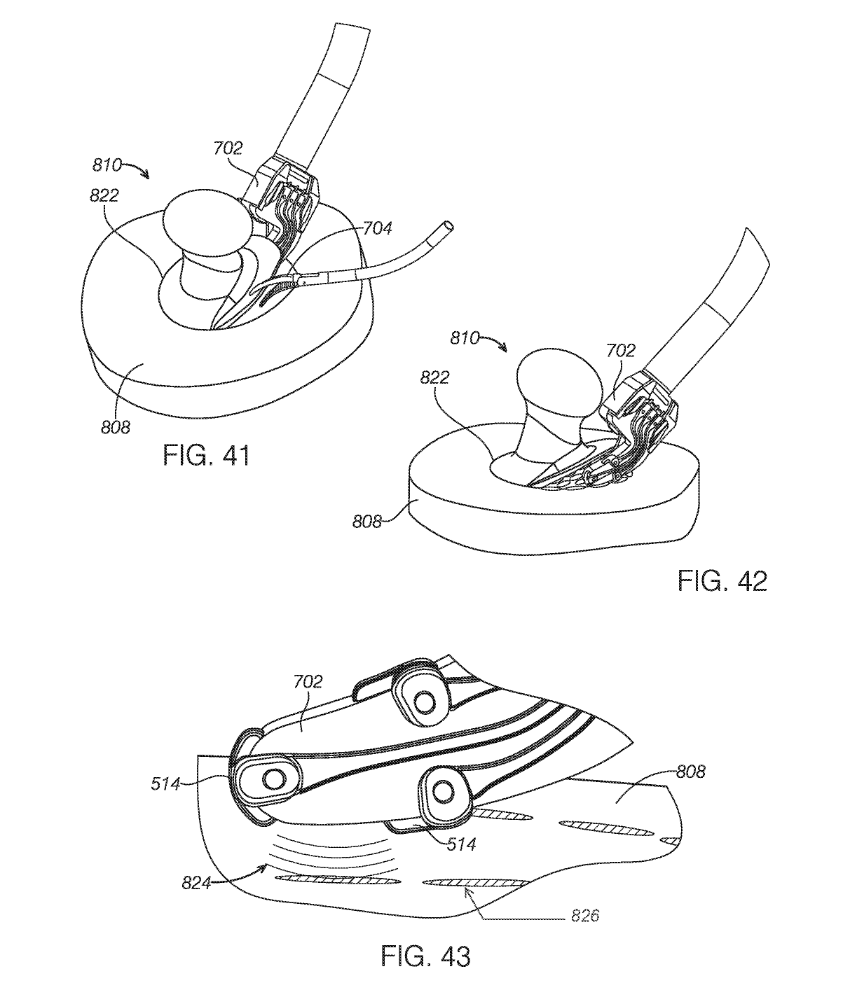

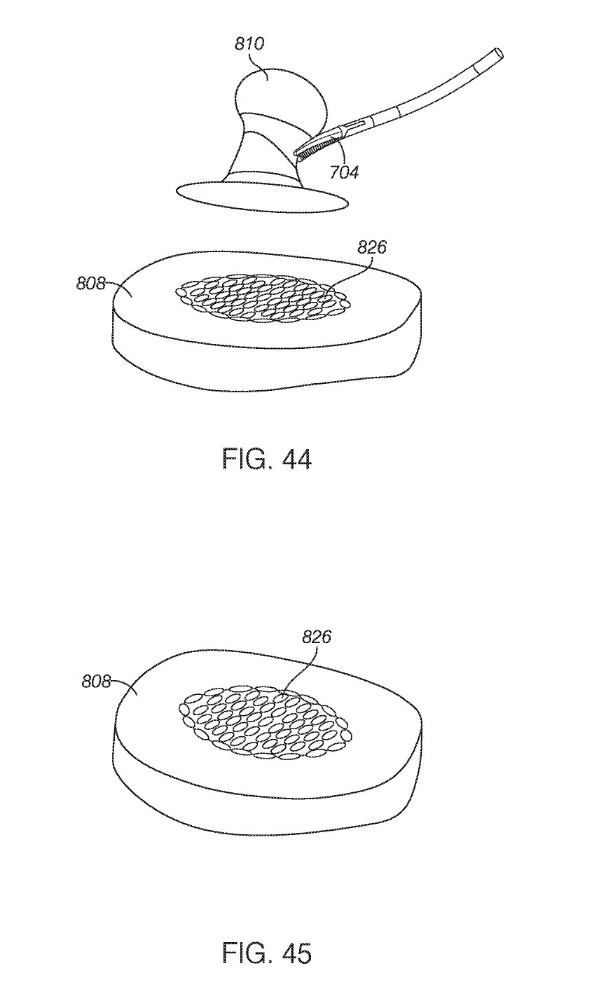

Referring to FIGS. 39-45, the overall steps of an exemplary polyp resection are shown and described. FIG. 39 depicts a typical resection path 822 followed by micro-shears 702. Path 822 extends in a generally circular path around the outside of base portion 816 of polyp 810. FIG. 40 shows micro-shears 702 beginning to cut along resection path 822 and a layer of submucosa starting to lift. In some implementations, the fixed tip of micro-shears 702 and/or the electrodes thereon are used to make an initial puncture through the layer to be cut so that fixed arm or horn 512 (shown in FIG. 11) can get beneath the layer during the cutting procedure. FIG. 41 depicts micro-shears 702 cutting further around polyp base 816 along path 822, and graspers 704 being used to lift the cut tissue. FIG. 42 shows micro-shears 702 cutting around the opposite side of polyp 810 from the initial cutting direction. FIG. 43 is an enlarged view of the tip of micro-shears 702 shown in FIG. 42. RF energy 824 is depicted emanating from electrodes 514 to create intermittent coagulated portions 826 along the tissue. FIG. 44 shows polyp 810 being lifted away from the intestinal wall 808 after it has been completely cut free. FIG. 45 shows the final resection site after the polyp has been completely removed and the underlying tissue has been coagulated by the micro-shears.

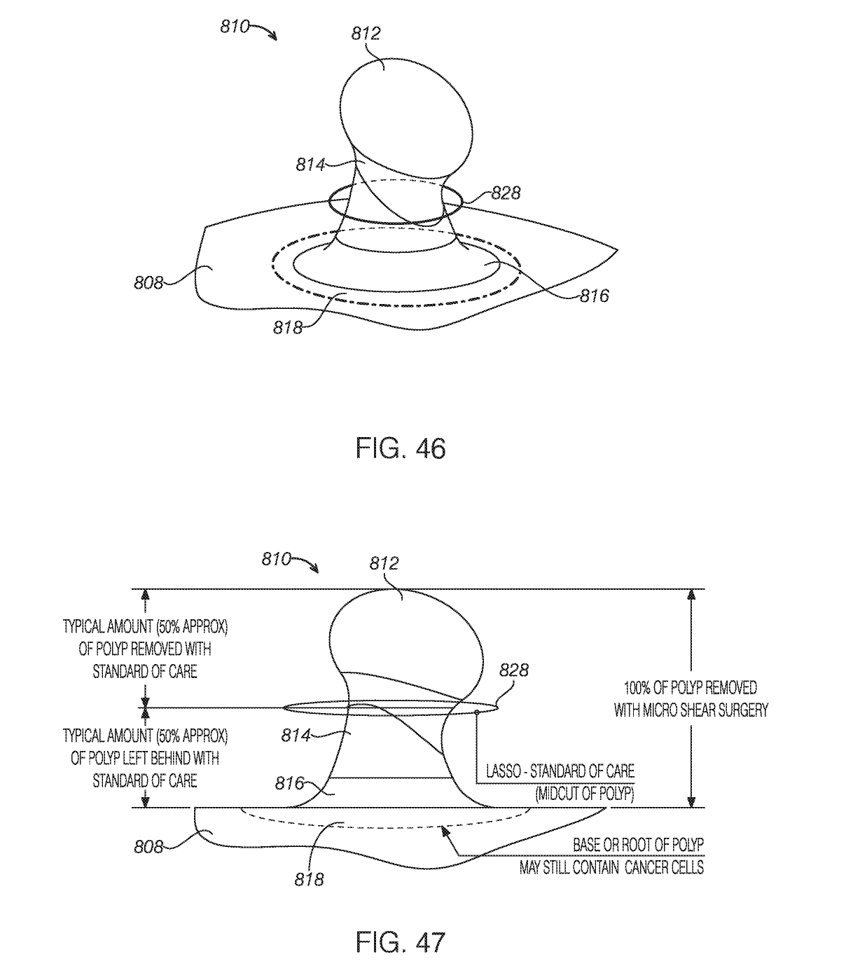

Referring to FIGS. 46-47, a comparison between conventional polyp or tumor resection techniques with the systems and methods disclosed herein is show and described. As shown in FIG. 46, the current standard of care involves encircling the reduced diameter body portion 814 of polyp 810 with a lasso or snare 828 delivered through a colonoscope. Electric current or RF energy may be applied to snare 828 to aid in cutting through the body of polyp 810 with snare 828 and to provide cauterization to the remaining tissue. A major drawback to this current standard of care is that it is difficult to place snare 828 close to the base 816 of polyp 810 and therefore a significant portion of the body 814 of polyp 810 is left behind. Typically, only 50% of the height of a polyp is removed with current practices, as depicted in FIG. 47. The remaining 50% left intact may still contain cancer cells. Even if snare 828 can be placed low on polyp 810, the remaining base 816 and root portion 818 may still contain cancer cells, and cannot be removed for biopsy leaving this portion of the polyp or tumor in question. As shown in FIG. 47, 100% of polyp 810 can generally be removed with the micro-shear systems and methods disclosed herein. Additionally, one of the most common post-polypectomy complications currently is bleeding. The micro-shear systems and methods disclosed herein provide effective cauterization/coagulation/sealing capabilities to address this complication. Tattooing of a polypectomy or tumor site may also be accomplished using the disclosed micro-shear systems to facilitate future surgery or endoscopic surveillance.

In view of the teachings herein, many further embodiments, alternatives in design and uses of the embodiments disclosed herein will be apparent to those of skill in the art. As such, it is not intended that the invention be limited to the particular illustrative embodiments, alternatives, and uses described above but instead that it be defined by the claims presented hereafter.

1: A powered scissors device comprising:
a distal housing having a fixed cutting arm located at a distal end thereof and extending beyond an interior portion of the distal housing;
an elongate member coupled to a proximal end of the distal housing and configured to introduce the distal housing to a target tissue site of the subject, the elongate member comprising an outer tube and an inner drive tube rotatably mounted within the outer tube;
a rotatable blade, rotatably mounted to the distal housing, having one or more cutting elements configured to cooperate with the fixed arm to shear tissue therebetween wherein the tissue that is being sheared is not located in an interior of the distal housing at the time of shearing;
a crown gear located at a distal end of the inner drive tube; and
a first spur gear configured to inter-engage with the crown gear and coupled with the rotatable blade to allow the crown gear to drive the rotatable blade through a rotation of at least one full revolution,
wherein every cutting edge of the one or more cutting elements remains in a single, common cutting plane as the one or more cutting elements rotate about an axis of rotation, thereby allowing the rotatable blade to make a single cutting line through the tissue.

2: The device of claim 1, wherein the rotatable blade has an axis of rotation that is perpendicular to an axis of rotation of the inner drive tube.

3: The device of claim 1, wherein the rotatable blade is partially located within a slot formed within the distal housing such that the at least one cutting element is covered by the distal housing during at least half of its rotation about an axis of rotation of the rotatable blade.

4: The device of claim 1, wherein the rotatable blade has multiple cutting elements, each of the cutting elements having a cutting edge configured to cooperate with a cutting edge of the fixed arm to shear tissue therebetween.

5. (canceled)

6: The device of claim 1, wherein the cutting element is shorter than the fixed arm.

7: The device of claim 1, wherein the cutting element has a top side and a bottom side, is flat on the top side, and has a cutting bevel provided along the bottom side.

8: The device of claim 1, wherein the cutting element has a cutting edge that is curved, and the fixed arm has a cutting edge that is curved in the same direction.

9: The device of claim 8, wherein the cutting edges of the cutting element and the fixed arm are curved in an outward direction trailing away from a direction of rotation of the cutting element.

10: The device of claim 8, wherein the cutting edge of the cutting element has a smaller radius of curvature than a radius of curvature of the cutting edge of the fixed arm.

11: The device of claim 1, wherein the fixed cutting arm is provided with at least one radio frequency electrode.

12: The device of claim 11, wherein the fixed cutting arm is provided with at least one pair of bipolar radio frequency electrodes.

13: The device of claim 11, wherein the fixed cutting arm comprises at least one conductive trace formed on a dielectric substrate and electrically connected to the at least one electrode.

14: The device of claim 13, wherein the fixed cutting arm further comprises at least one electrical connector located on the dielectric substrate and electrically connected to the at least one conductive trace.

15: The device of claim 14, wherein the at least one electrical connector comprises a plurality of locking barbs configured to retain a mating electrical pin.

16: The device of claim 14, wherein the at least one electrical trace and the at least one electrical connector have both been formed together by a material additive process.

17: The device of claim 14, wherein the fixed cutting arm is removable from the distal housing by releasing at least one locking member and sliding the fixed cutting arm out of the distal housing.

18: The device of claim 11, wherein the at least one electrode comprises three surfaces that extend in three mutually orthogonal directions.

19: The device of claim 11, wherein the at least one electrode comprises an outer working surface having texturing features, thereby increasing an overall surface area of the at least one electrode without increasing dimensions of the outer working surface.

20: A method of submucosa resection of colon polyps, the method comprising:
advancing a distal end of a colonoscope into a patient's colon toward a target polyp;
extending micro-shears from the distal end of the colonoscope, wherein the micro-shears have a maximum lateral cross-section that fits within a 10 mm circle, the micro-shears comprising a distal housing having a fixed cutting arm located thereon, a rotatable blade rotatably mounted to the distal housing, a crown gear located at a distal end of an inner drive tube, and a first spur gear configured to inter-engage with the crown gear and coupled with the rotatable blade;
grasping the target polyp with a grasper that can move independently of the movement of the micro-shears;
driving the rotatable blade with the inner drive tube and the crown gear such that the blade rotates at least one full revolution;
applying the rotatable blade to tissue adjacent to the target polyp, while using the grasper to lift the polyp away from surrounding tissue, such that the rotatable blade and the fixed cutting arm cooperate to shear tissue therebetween wherein the tissue that is being sheared is not within an interior of the distal housing during shearing, and such that the rotatable blade and the fixed cutting arm follow a generally circular resection path around a base portion of the target polyp to cut a layer of submucosa with a single cutting line through the tissue; and
removing the target polyp through the colonoscope, including removing a head portion, a body portion, a base portion, and a root portion of the target polyp.

21: The method of claim 20, wherein the rotatable blade comprises a plurality of cutting elements configured to cooperate with the fixed cutting arm to shear tissue therebetween.

22: The method of claim 21, wherein each of the cutting elements has at least one cutting edge, and wherein each of the cutting edges of the cutting elements remains in a single, common cutting plane as the plurality of cutting elements rotate about a common axis of rotation.

23: The method of claim 20, wherein the head, body, base and root portions of the target polyp are lifted away from the adjacent tissue and removed through the colonoscope in a single piece.

24: The method of claim 23, wherein the grasper is manipulated through the colonoscope to hold the target polyp while the micro-shears cut the layer of submucosa around the base portion of the target polyp, and wherein the grasper is used to lift the target polyp away from the adjacent tissue.

25: The method of claim 20, wherein the step of applying the rotatable blade to the tissue adjacent to the polyp comprises making an initial puncture in the adjacent tissue with the fixed cutting arm of the micro-shears so that the fixed cutting arm gets beneath a portion of the adjacent tissue.

26: The method of claim 25, wherein the fixed cutting arm of the micro-shears comprises at least one radio frequency electrode that is used to assist in making the initial puncture.

27: The method of claim 20, further comprising coagulating the adjacent tissue using at least one radio frequency electrode located on the fixed cutting arm of the micro-shears.

28: The method of claim 27, wherein the at least one electrode comprises three surfaces that extend in three mutually orthogonal directions.

29: The method of claim 27, wherein the at least one electrode comprises an outer working surface having texturing features, thereby increasing an overall surface area of the at least one electrode without increasing dimensions of the outer working surface.

30: A method of submucosa resection of colon polyps, the method comprising:
advancing a distal end of a colonoscope into a patient's colon toward a target polyp;
extending micro-shears from the distal end of the colonoscope, wherein the micro-shears have a maximum lateral cross-section that fits within a 10 mm circle, the micro-shears comprising a distal housing having a fixed cutting arm located thereon, a rotatable blade rotatably mounted to the distal housing, a crown gear located at a distal end of an inner drive tube, and a first spur gear configured to inter-engage with the crown gear and coupled with the rotatable blade;
driving the rotatable blade with the inner drive tube and the crown gear such that the blade spins a plurality of revolutions in a constant direction of rotation about an axis of rotation, and wherein the rotatable blade is partially located within a slot formed within the distal housing such that a plurality of cutting portions of the blade are covered by the distal housing during at least half of each rotation about the axis of rotation;
making an initial puncture in the tissue adjacent to the target polyp using a pair of radio frequency electrodes located on the fixed cutting arm of the micro-shears so that the fixed cutting arm gets beneath a portion of the adjacent tissue, wherein each of the pair of electrodes comprises three surfaces that extend in three mutually orthogonal directions, and wherein each of the pair of electrodes comprises an outer working surface having texturing features, thereby increasing an overall surface area of the electrode without increasing dimensions of the outer working surface;
applying the rotatable blade to the adjacent tissue such that a plurality of cutting elements located on the rotatable blade cooperate with the fixed cutting arm to shear tissue therebetween wherein the tissue that is being sheared is not located in an interior of the distal housing at the time of shearing, wherein each of the cutting elements has at least one cutting edge, and wherein each of the cutting edges of the cutting elements remains in a single, common cutting plane as the plurality of cutting elements rotate about a common axis of rotation, wherein the rotatable blade and the fixed cutting arm follow a generally circular resection path around a base portion of the target polyp to cut a layer of submucosa with a single cutting line through the tissue;
manipulating graspers, that are independently controllable relative to the micro-shears, through the colonoscope to hold and lift the target polyp while the micro-shears cut the layer of submucosa around the base portion of the target polyp;
lifting the target polyp with the graspers away from the adjacent tissue after cutting is complete;
removing the target polyp through the colonoscope, including removing a head portion, a body portion, a base portion, and a root portion of the target polyp in a single piece; and
coagulating the adjacent tissue using the pair of electrodes located on the fixed cutting arm of the micro-shears.
